(19) United States
(12) Patent Application Publication
(10) Pub. No.: US 2022/0033917 A1
(43) Pub. Date: Feb. 3, 2022

(54) CANCER CELL METHYLATION MARKERS AND USE THEREOF

(71) Applicants: YISSUM RESEARCH DEVELOPMENT COMPANY OF THE HEBREW UNIVERSITY OF JERUSALEM LTD., Jerusalem (IL); HADASIT MEDICAL RESEARCH SERVICES & DEVELOPMENT LTD., Jerusalem (IL)

(72) Inventors: Yuval DOR, Jerusalem (IL); Ruth SHEMER, Mevasseret Zion (IL); Benjamin GLASER, Jerusalem (IL); Tomer KAPLAN, Jerusalem (IL); Joshua MOSS, Jerusalem (IL); Netanel LOYFER, Jerusalem (IL); Daniel NEIMAN, Bnei Dekalim (IL)

(21) Appl. No.: 17/503,666

(22) Filed: Oct. 18, 2021

Related U.S. Application Data
(63) Continuation of application No. PCT/IL2020/050451, filed on Apr. 16, 2020.
(60) Provisional application No. 62/835,069, filed on Apr. 17, 2019.

(51) Int. Cl. C12Q 1/6886

(52) U.S. Cl. CPC C12Q 1/6886; C12Q 1/6869; C12Q 2600/154

(57) ABSTRACT

Methods of detecting DNA from a cancerous cell comprising receiving measurements of DNA methylation in at least one genomic region are provided. Arrays comprising at least 10 methylation specific oligonucleotides, wherein the methylation specific oligonucleotides are each reverse complementary to a genomic region are also provided.

### CROSS REFERENCE TO RELATED APPLICATIONS

This application is a Bypass Continuation of PCT Patent Application No. PCT/IL2020/050451 having International filing date of Apr. 16, 2020, which claims the benefit of priority of U.S. Provisional Patent Application No. 62/835,069, filed Apr. 17, 2019, the contents of which are all incorporated herein by reference in their entirety.

### FIELD OF INVENTION

The present invention is in the field of cancer markers.

### BACKGROUND OF THE INVENTION

Detection of cancerous cells is essential for early disease identification, distinguishing malignant and non-malignant growths, tracking treatment effectiveness, and monitoring for residual disease. Each of these monitoring modalities requires certainty in identifying cancer cells and distinguishing them from non-cancer cells. Beyond this detection of very low amounts of DNA from cancer cells facilitates superior detection and more precise identification of therapeutic effects. Although mutational changes to the DNA sequence of cancer cells are common, they are heterogenous and not always known. Further, these mutations occur in healthy cells as well complicating their use as markers for cancer. Epigenetic cancer makers, most notably methylation marks, are emerging as a reliable marker to use in place of mutations.

Cell death often involves the release of short DNA fragments into the blood, known as circulating cell-free DNA (cfDNA). Liquid biopsy—the analysis of cfDNA in the plasma—has recently emerged as a powerful diagnostic tool for cancer, allowing the identification of genetic mutations in DNA molecules originating from the tumor. Since liquid biopsy is non-invasive, it allows for very early cancer detection, facilitates monitoring of disease progression and treatment efficacy, and can be used to screen for residual disease after a successful treatment. However, due to the typically low number of informative driver mutations in cancer, such approaches are constrained by tumor size and might not detect tumors smaller than 10 cm³. Generally, identifying very rare DNA fragments greatly limit the effectiveness of most cancer monitoring modalities. New methods of cancer identification, that are highly sensitive and highly cancer-specific are greatly needed.

### SUMMARY OF THE INVENTION

The present invention provides methods of detecting DNA from a cancerous cell, comprising measuring DNA methylation in at least one informative genomic region and assigning a sample as comprising DNA from a cancer cell when the region bares a cancer-specific methylation mark. Arrays comprising at least 10 methylation specific oligonucleotides, wherein the methylation specific oligonucleotides are each reverse complementary to a genomic region are also provided.

According to a first aspect, there is provided a method of detecting DNA from a cancerous cell in a sample, the method comprising:
a. receiving DNA methylation measurements of DNA from a sample in at least one genomic region comprising CpG dinucleotides, wherein the at least one genomic region is selected from a region provided in Table 1 and Table 2; and
b. assigning a sample as comprising DNA from a cancerous cell when the region comprises a cancer-specific methylation pattern; thereby detecting DNA from a cancerous cell is a sample comprising DNA.

According to some embodiments, the receiving comprises providing a sample comprising DNA and measuring DNA methylation of the DNA in the at least one genomic region selected from a region provided in Table 1 and Table 2.

According to some embodiments, the sample is selected from a blood sample, a bodily fluid sample, a tissue sample and a tumor sample.

According to some embodiments, the sample is a bodily fluid sample, the DNA is cell-free DNA, and wherein the providing comprises providing a bodily fluid and isolating the cfDNA from the bodily fluid.

According to some embodiments, the biological fluid is selected from blood, plasma, serum, urine, feces, cerebral spinal fluid, lymph, tumor fluid and breast milk.

According to some embodiments, the biological fluid is peripheral blood.

According to some embodiments, the DNA from a cancerous cell is less than 0.1% of the cfDNA.

According to some embodiments, the sample is obtained from a subject and the method is for detecting cancer in the subject.

According to some embodiments, the method further comprises administering an anti-cancer therapy to a subject for whom cancer is detected.

According to some embodiments, the measurements of DNA methylation comprises measurement of bisulfite converted DNA.

According to some embodiments, the measurements comprise measurements from performing a methylome array or chip on the bisulfite converted DNA, or sequencing the bisulfate converted DNA.

According to some embodiments, the measurements are from performing methylation specific PCR.

According to some embodiments, the cancer-specific methylation pattern is hypermethylation of at least one genomic region provided in Table 1.

According to some embodiments, the cancer-specific methylation pattern is methylation of a central CpG of the at least one genomic region provided in Table 1.

According to some embodiments, the cancer-specific methylation pattern further comprises methylation of at least one other CpG of the at least one genomic region.

According to some embodiments, the hypermethylation comprises methylation of at least 5 CpGs within the region.

According to some embodiments, the cancer-specific methylation pattern is hypomethylation of at least one region provided in Table 2.

According to some embodiments, the cancer-specific methylation pattern is unmethylation of a central CpG of the at least one genomic region provided in Table 2.

According to some embodiments, the cancer-specific methylation pattern further comprises unmethylation of at least one other CpG of the at least one genomic region.

According to some embodiments, the hypermethylation comprises methylation of at least 5 CpGs within the region.

According to some embodiments, the at least one region is a region from 100 nucleotides upstream of a central CpG provided in Table 1 and 2 to 100 nucleotides downstream of the central CpG.

According to some embodiments, the cancer is selected from breast cancer, cervical cancer, endocervical cancer, colon cancer, lymphoma, esophageal cancer, brain cancer, head and neck cancer, renal cancer, meningeal cancer, glioma, glioblastoma, Langerhans cell cancer, lung cancer, mesothelioma, ovarian cancer, pancreatic cancer, neuroendocrine cancer, prostate cancer, skin cancer, stomach cancer, tenosynovial cancer, thyroid cancer, uterine cancer, and testicular cancer.

According to some embodiments, the cancer-specific methylation pattern is a specific cancer-specific methylation pattern, and the cancer and region match based on the methylation levels provided in Table 3.

According to another aspect, there is provided an array, comprising at least 10 methylation specific oligonucleotides, wherein the at least 10 methylation specific oligonucleotides each is reverse complementary to a sequence of a genomic region provided in Table 1 and Table 2.

According to some embodiments, the array further comprises a solid support, wherein the at least 10 methylation specific oligonucleotides are immobilized to the solid support.

According to some embodiments, a methylation specific oligonucleotide only hybridizes in the presence of methylation or only binds in the absence of methylation.

According to some embodiments, a methylation specific oligonucleotide is reverse complementary
a. to a sequence of a region from Table 1 and is not complementary to sequence of a region from Table 1 wherein a cytosine of a CpG residue is converted to a thymine, or
b. to a sequence of a region from Table 2 wherein a cytosine of a CpG residue is converted to a thymine and is not complementary to a sequence of a region from Table 2.

According to some embodiments, the array comprises at least 100 oligonucleotides.

According to some embodiments, the array comprises a plurality of oligonucleotides that are reverse complementary to a region.

According to some embodiments, the array comprises at least one methylation specific oligonucleotide reverse complementary to each region in Table 1 and Table 2.

According to some embodiments, a methylation specific oligonucleotide reverse complementary to a region is reverse complementary to a central CpG of the region.

According to some embodiments, the methylation specific oligonucleotide is reverse complementary to a region from 100 nucleotides upstream of a central CpG provided in Table 1 and 2 to 100 nucleotides downstream of the central CpG.

According to another aspect, there is provided a kit comprising an array of the invention and at least one reagent for amplification of a target DNA molecule hybridized to an oligonucleotide of the array.

According to some embodiments, the reagent is selected from a polymerase, a forward primer, a reverse primer, an adapter, and a pool of free nucleotides.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A-C: Differential DNA methylation patterns. (1A) Some genomic regions show cancer-specific methylation along multiple CpG sites (dark grey). These patterns are absent from healthy plasma samples and can serve as minimally invasive pan-cancer biomarkers. (1B-C) Line graphs showing that by combining 20 multiple genomic regions, the sensitivity and specificity of “liquid biopsy” tests is dramatically improved, to near 100%, at ˜0.1% load of circulating cancer cfDNA. The computer simulation is based on high methylation (85%) in cancer, compared to low (15%) in healthy cells. For simplicity, it is also assumed that neighboring CpGs are de/methylated independently of each other. According to that model, the likelihood of the event “five methylated CpGs” in a given CpG block, in cancer is 0.44371 (=0.855), compared to 7.6e-05 (=0.155) for normal cells (about ˜6000 times less likely). By integrating the prior probability of tumor DNA compared to normal DNA in the plasma, one can then apply Bayes' law and infer the conditional probability of cancer given such an event. (1C) shows integration of 20 sites with 8 CpGs each, that are sufficient to detect loads of 0.1%-1% of circulating tumor DNA in high sensitivity (>98%) and specificity (>99.99%).

FIG. 2: Dot plots of methylation levels in various cancer, matched healthy tissues, and healthy tissues/cell types. The order of the samples from left to right is: cancers-BLCA, BRCA, CESC, COAD, ESCA, GBM, HNSC, KIRC, KIRP, LIHC, LUAD, LUSC, PAAD, PCPG, PRAD, READ, SARC, SKCM, STAD, HCA, THYM, UCEC; healthy tissues/cell types-neutrophils, monocytes, erythroid progenitors, CD4+ T cells, CD8+ T cells B-cells, NK-cells, Eosinophils, vascular endothelial cells, hepatocytes.

FIG. 3: Table 3 showing methylation values for 87 regions in cancer samples, matching healthy samples and healthy tissues/cell types.

FIGS. 4A-D: (4A) A dot plot of methylation levels in various cancer, matched healthy tissues, and healthy tissues/cell types at central CpG cg00100121. The order of the samples from left to right is: cancers-BLCA, BRCA, CESC, COAD, ESCA, GBM, HNSC, KIRC, KIRP, LIHC, LUAD, LUSC, PAAD, PCPG, PRAD, READ, SARC, SKCM, STAD, HCA, THYM, UCEC; healthy tissues/cell types-neutrophils, monocytes, erythroid progenitors, CD4+ T cells, CD8+ T cells B-cells, NK-cells, Eosinophils, vascular endothelial cells, hepatocytes. (4B-D) Bisulfite modified reads of the region 250 nucleotides upstream and downstream of central CpG cg00100121 in (4B) cancer samples, (4C) healthy tissue and (4D) cfDNA from blood of healthy donors.

FIGS. 5A-D: (5A) A dot plot of methylation levels in various cancer, matched healthy tissues, and healthy tissues/cell types at central CpG cg00002719. The order of the samples from left to right is: cancers-BLCA, BRCA, CESC, COAD, ESCA, GBM, HNSC, KIRC, KIRP, LIHC, LUAD, LUSC, PAAD, PCPG, PRAD, READ, SARC, SKCM, STAD, HCA, THYM, UCEC; healthy tissues/cell types-neutrophils, monocytes, erythroid progenitors, CD4+ T cells, CD8+ T cells B-cells, NK-cells, Eosinophils, vascular endothelial cells, hepatocytes. (5B-D) Bisulfite modified reads of the region 250 nucleotides upstream and downstream of central CpG cg00002719 in (5B) cancer samples, (5C) healthy tissue and (5D) cfDNA from blood of healthy donors.

FIGS. 6A-D: (6A) A dot plot of methylation levels in various cancer, matched healthy tissues, and healthy tissues/cell types at central CpG cg24748548. The order of the samples from left to right is: cancers-BLCA, BRCA, CESC, COAD, ESCA, GBM, HNSC, KIRC, KIRP, LIHC, LUAD, LUSC, PAAD, PCPG, PRAD, READ, SARC, SKCM, STAD, HCA, THYM, UCEC; healthy tissues/cell types-neutrophils, monocytes, erythroid progenitors, CD4+ T cells, CD8+ T cells B-cells, NK-cells, Eosinophils, vascular endothelial cells, hepatocytes. (6B-D) Bisulfite modified reads of the region 250 nucleotides upstream and downstream of central CpG cg24748548 in (6B) cancer samples, (6C) healthy tissue and (6D) cfDNA from blood of healthy donors.

FIGS. 7A-B: (7A) A bar chart of accumulated cancer specific methylation reads in healthy samples and tumor samples. (7B) A bar chart of accumulated cancer specific methylation reads in cfDNA samples from healthy and breast cancer patients.

### DETAILED DESCRIPTION OF THE INVENTION

The present invention, in some embodiments, provides methods of detecting DNA from a cancerous cell in a sample and arrays for doing same.

By a first aspect, there is provided a method of detecting DNA from a cancerous cell in a sample, the method comprising: receiving DNA methylation measurements of DNA from the sample in at least one genomic region and assigning a sample as comprising DNA from a cancerous cell when the region comprises a cancer-specific methylation pattern, thereby detecting DNA from a cancerous cell in a sample.

In some embodiments, the method is an in vitro method. In some embodiments, the method is an ex vivo method. In some embodiments, the method is a diagnostic method. In some embodiments, the method is a non-invasive method. In some embodiments, the sample if from a subject. In some embodiments, the method is for diagnosing cancer in a subject. In some embodiments, the method is for detecting cancer in a subject. In some embodiments, the detection is early detection. In some embodiments, the detection is detection with increases sensitivity. In some embodiments, the detection is detection with increased specificity. In some embodiments, the increase is as compared to cancer detection by a cancer specific mutation. In some embodiments, the increase is as compared to cancer detection by methylation of a region that is not a region of the invention. In some embodiments, the increase is as compared to any other method of cancer detection other than that of the invention. In some embodiments, the detection is detection of a tumor smaller than 10 cubic cm. In some embodiments, the detection is detection of less than 0.1% tumor DNA in a cfDNA sample. In some embodiments, the detection is detection of less than 1, 0.5, 0.1, 0.05, 0.01, 0.005 or 0.001% tumor DNA in a cfDNA sample. Each possibility represents a separate embodiment of the invention. In some embodiments, the method is for detecting residual disease in a subject. In some embodiments, the disease is cancer. In some embodiments, the method is for detecting death of cancer cells in a subject. In some embodiments, the method is for monitoring disease progression in a subject. In some embodiments, the method is for monitoring treatment efficacy in a subject. In some embodiments, increase cancer cell death indicates increased efficacy of a treatment. In some embodiments, absence or decrease in cancer cell cfDNA indicates efficacy of a treatment.

In some embodiments, the method further comprises treating the cancer. In some embodiments, the method further comprises treating the detected cancer. In some embodiments, the treating is administering an anticancer therapy. In some embodiments, the treating is reinitiated a discontinued therapy. In some embodiments, the reinitiating is after discovery of residual disease after an effective therapy. In some embodiments, the treating is continuing a treatment found to effective by a method of the invention. In some embodiments, the therapy is radiation. In some embodiments, the therapy is chemotherapy. In some embodiments, the therapy is immunotherapy. Any anti-cancer therapy known in the art may be used.

In some embodiments, the sample comprises DNA. In some embodiments, the sample comprises cells. In some embodiments, the sample comprises cell free DNA. In some embodiments, the DNA is sheared DNA. In some embodiments, the DNA is fragmented DNA. In some embodiments, the DNA is caspase cleaved DNA. In some embodiments, the sample comprises lysed cells. In some embodiments, the sample comprises apoptotic cells. In some embodiments, the sample comprises dead cells. In some embodiments, the sample comprises necrotic cells. In some embodiments, the sample is a blood sample. In some embodiments, the sample is a plasma sample. In some embodiments, the sample is a serum sample. In some embodiments, the sample is a bodily fluid sample. In some embodiments, the sample is a bodily fluid sample and the DNA is cfDNA. In some embodiments, the sample is a tissue sample. In some embodiments, the sample is a tumor sample. In some embodiments, the sample is a biopsy. In some embodiments, the sample is a liquid biopsy. In some embodiments, the sample is from a growth whose malignancy is unknown. In some embodiments, the bodily fluid is selected from blood, plasma, serum, urine, feces, cerebral spinal fluid, lymph, tumor fluid and breast milk. In some embodiments, the blood is peripheral blood.

In some embodiments, the sample is from a subject. In some embodiments, the subject is a mammal. In some embodiments, the mammal is a human. In some embodiments, the subject is at risk for developing cancer. In some embodiments, the subject is suspected of having cancer. In some embodiments, the subject is genetically predisposed to cancer. In some embodiments, the subject has a growth of unknown character. In some embodiments, the growth has unknown malignancy. In some embodiments, the growth in not known to be benign. In some embodiments, the subject is a healthy subject. In some embodiments, the subject is providing a routine blood sample. In some embodiments, the subject is already diagnosed with cancer by means other than those of the present invention. In some embodiments, the cancer diagnosed subject has begun cancer treatment. In some embodiments, the subject has cancer. In some embodiments, the subject is undergoing cancer treatment. In some embodiments, the subject has cancer that is in remission. In some embodiments, the subject had cancer that has been cured. In some embodiments, the subject had cancer which is now undetectable. In some embodiments, the subject has completed a regimen of cancer treatment. In some embodiments, the subject is at risk for cancer return. In some embodiments, the subject is at risk for cancer relapse.

As used herein, the term “cancer” refers to any disease characterized by abnormal cell growth. In some embodiments, cancer is further characterized by the potential or ability to invade to other parts of the body beyond the part where the abnormal cell growth originated. In some embodiments, cancer is selected from breast cancer, cervical cancer, endocervical cancer, colon cancer, lymphoma, esophageal cancer, brain cancer, head and neck cancer, renal cancer, meningeal cancer, glioma, glioblastoma, Langerhans cell cancer, lung cancer, mesothelioma, ovarian cancer, pancreatic cancer, neuroendocrine cancer, prostate cancer, skin cancer, stomach cancer, tenosynovial cancer, tongue cancer, thyroid cancer, uterine cancer, and testicular cancer. In some embodiments, the cancer is selected from the types of cancer listed in Table 3. In some embodiments, the cancer is the same type of cancer as the cancer samples in Table 3. In some embodiments, the cancer is breast cancer. In some embodiments, the cancer is pancreatic cancer. In some embodiments, the cancer is lung cancer. In some embodiments, the cancer is hepatic cancer. In some embodiments, the cancer is colon cancer. In some embodiments, the cancer is tongue cancer. In some embodiments, the cancer is carcinoma. In some embodiments, the cancer is a glioma. In some embodiments, the cancer is a melanoma. In some embodiments, the cancer is a solid cancer. In some embodiments, the cancer is a blood cancer. In some embodiments, the cancer is a tumor.

In some embodiments, the method comprises receiving DNA methylation measurements of DNA from a sample. In some embodiments, receiving comprises providing a sample comprising DNA. In some embodiments, the method comprises extracting DNA from the sample. In some embodiments, the method comprises isolating DNA from the sample. In some embodiments, the method comprises measuring methylation in at least one genomic region of the DNA. In some embodiments, the method comprises measuring methylation in at least one genomic region. In some embodiments, measurements of DNA methylation comprise measurement of bisulfite converted DNA. In some embodiments, measuring DNA methylation comprise measuring bisulfite converted DNA. In some embodiments, the method comprises bisulfite conversion of the DNA in the sample. In some embodiments, the method comprises performing bisulfite conversion of the DNA. In some embodiments, the method comprises bisulfite conversion of the genomic region. In some embodiments, measurements of DNA methylation comprise measurements from performing a methylome array or chip. In some embodiments, the measurements are methylome array or chip measurements. In some embodiments, measuring comprises performing a methylome array of chip. In some embodiments, the methylome array or chip is performed on bisulfite converted DNA. In some embodiments, the methylome array or chip is performed on DNA from the sample. In some embodiments, the measurements are sequencing measurements. In some embodiments, the measurements are from sequencing. In some embodiments, the measuring comprises sequencing. In some embodiments, the sequencing is sequencing of bisulfite converted DNA. In some embodiments, the sequencing is sequencing of DNA in the sample. In some embodiments, the sequencing is sequencing of the genomic region. In some embodiments, the sequencing is next generation sequencing. In some embodiments, the sequencing is deep sequencing. In some embodiments, the sequencing is massively parallel sequencing. In some embodiments, the measurements are from performing methylation specific PCR. In some embodiments, the measurements are of methylation specific PCR. In some embodiments, measuring comprises performing methylation specific PCR. In some embodiments, methylation specific PCR is multiplex methylation specific PCR. In some embodiments, the methylome chip/array is Twist targeted methylation sequencing.

In some embodiments, sequencing comprises ligating adapters to the DNA. In some embodiments, the adapters are ligated before bisulfite conversion. In some embodiments, the adapters are ligated after bisulfite conversion. In some embodiments, method comprises isolating DNA comprising a fragment from a region provided in Table 1 and Table 2. In some embodiments, the isolating comprises hybridizing an oligonucleotide to the region. In some embodiments, the oligonucleotide is an oligonucleotide of the invention. In some embodiments, the oligonucleotide is immobilized to a solid support. In some embodiments, the oligonucleotide is a synthetic oligonucleotide. In some embodiment, the solid support is a synthetic solid support. In some embodiments, the solid support is a non-natural solid support. In some embodiments, the solid support is a man-made solid support. In some embodiments, sequencing comprises capturing a target molecule. In some embodiments, the target molecule is a DNA molecule. In some embodiments, the target molecule comprises at least a fragment of a genomic region. In some embodiments, the target molecule is a bisulfite converted DNA. In some embodiments, the target molecule is a cfDNA molecule. By isolating the regions of interest before sequencing the sensitivity and specificity of the assay can be increased and the noise can be reduced. In this way only the informative samples are analyzed. It is of course also possible to sequence all of the DNA in the sample and diagnose only based on the informative regions.

In some embodiments, sequencing further comprises reverse transcribing (RT) the target molecule. In some embodiments, the oligonucleotide is the primer for RT. In some embodiments, the method comprises contacting the target molecule with a primer for RT. In some embodiments, the method comprises amplifying the target molecule. In some embodiments, the target molecule is bisulfite converted before amplification. As most amplification methods do not retain methylation of CpG dinucleotides, the amplification is often performed after bisulfite conversion. In some embodiments, the amplification further comprises contacting a reverse transcribed strand with a reverse primer. In some embodiments, amplification is with a forward and reverse primer.

Bisulfite conversion of DNA is a standard biochemical assay. A standard protocol can be found in “Bisulfite Sequencing of DNA”, Darst et al., 2010, Current Protocols in Molecular Biology, Chapter 7: unit 7.9.1-17, herein incorporated by reference in its entirety. In brief, bisulfite conversion comprises DNA denaturation, incubation with bisulfite at elevated temperature, removal of bisulfite by desalting, desulfonation of sulfonyl uracil adducts at alkaline pH and removal of the desulfonation solution. The result is that unmethylated cytosines are converted to thymine and methylated cytosines are unmodified. Thus, following bisulfite conversion any sequence that is identified with a cytosine indicates that cytosine was methylated in the DNA. These cytosines can be identified by sequencing, or by binding to a reverse complementary oligonucleotide that has a guanine to bind with the cytosine. If the reverse complementary sequence matches the converted sequence there will be hybridization and identification of the sequence. However, if the cytosine was converted to a thymine then the guanine cannot hybridize and there will not be binding. Alternatively, the oligonucleotide can be designed with an adenine to hybridize to a thymine at the location that was once cytosine. Thus, the oligonucleotide will only hybridize if the cytosine was unmethylated. These methylation specific oligonucleotides can be used for methylation specific PCR, or for methylome arrays or chips. The positioning of the potential methylated cytosine within the oligonucleotide is important as a 5′ location may still allow hybridization with a mismatch. Placement of the potentially methylated base at the 3′ end of the oligonucleotide increases the chance that lack of hybridization of the base will lead to a lack of hybridization of the whole oligonucleotide to the piece of DNA.

Methylome arrays/chips and kits are well known in the art and are commercially available. Illumina, for example, makes the MethylationEPIC BeadChip, and the Infinium MethylationEPC kit. Methylation specific PCR primers can be designed with the same software as standard primer design software, but by targeting the specific methylation site or region in question. Primer design software, such as Primer3 is well known in the art. Alternatively standard PCR, or quantitative PCR (qPCR) can be performed and then amplicon is sequenced. In some embodiments, the bisulfite conversion occurs before amplification. Exemplary primers for amplifying the regions of the invention are provided in Table 4

TABLE 4
Primers

| | | SEQ | | SEQ |
|---|---|---|---|---|
| | | ID | | ID |
| Marker Name | Primer1 | NO: | Primer2 | NO: |
| ANKS1B | gttgatgttt | 1 | tatatatcca | 2 |
| | gttatagggt | | aaaaaccaac | |
| | | | cc | |
| C17orf64TSS1500* | ttagggaaga | 3 | aaaaatactc | 4 |
| | aaaggtggtt | | aaaaaacccc | |
| C1orf114 | tatttttttt | 5 | ccataacaat | 6 |
| | gtttgtgtaa | | ataatcctaa | |
| | aatg | | ctacc | |
| C20orf103 | ggtttttttt | 7 | attctataaa | 8 |
| | ttggtagtga | | cccctaacta | |
| | | | aaa | |
| cg00002719* | agtgaagttg | 9 | aaaatttcac | 10 |
| | aggtttttaa | | aaccaacaca | |
| | gg | | ac | |
| cg00327669* | gagagaggtg | 11 | aaacatacac | 12 |
| | gttatggttg | | aacaaataac | |
| | | | acac | |
| cg00755470* | gttggaaggg | 13 | aaaacactac | 14 |
| | tgtaaggtgt | | acaatccccc | |
| cg01016662* | aaggaagttt | 15 | ctccccctac | 16 |
| | aggtgagata | | tactcctact | |
| | ggtt | | ctac | |
| cg02782369* | ggaattgtat | 17 | ctttaaaaat | 18 |
| | ttattttgga | | aaaaaaccat | |
| | gg | | tctac | |
| cg02996413* | atattttggg | 19 | tactaaacaa | 20 |
| | agatgagatg | | aacccctccc | |
| | g | | | |
| cg05289966* | ggagaggatg | 21 | ctctcccaaa | 22 |
| | atattattgg | | atattataaa | |
| | taata | | caata | |
| cg08042316* | gtgttaggag | 23 | ctaaaaactt | 24 |
| | attaagtttt | | accacaacta | |
| | gatt | | ataaac | |
| cg08042316 | agtaagagag | 25 | caaaaatcta | 26 |
| | ggatagagat | | aaaataacaa | |
| | agg | | aaaa | |
| cg08967106 | ggggaggtag | 27 | ccttaaaaaa | 28 |
| | tgatttaggt | | aaaaccaaaa | |
| | | | c | |
| cg10305311 | ggttgttagt | 29 | ttctccatct | 30 |
| | ttgaatttga | | acaactaacc | |
| | gt | | c | |
| cg12391352* | atagaaaggt | 31 | accataaata | 32 |
| | tgatgtttgt | | tatatccaaa | |
| | tata | | aaac | |
| cg13586420* | gaggttgata | 33 | cccttactac | 34 |
| | gaagataggg | | ataaaactaa | |
| | ag | | acc | |
| cg14038484* | ggagggtaaa | 35 | tcacacttct | 36 |
| | ggtttgtagg | | ttcccaataa | |
| | | | ac | |
| cg14160020* | tagggttagg | 37 | aaaactctaa | 38 |
| | agaaattatt | | taaaccaaat | |
| | gtt | | ctatt | |
| cg14203032* | ggtaaaattt | 39 | aaacactcac | 40 |
| | tttaaaagga | | ctaaaaacta | |
| | ata | | acc | |
| cg14440102 | tttatgttta | 41 | ccataattca | 42 |
| | ggatattaat | | ataaaaataa | |
| | ttattg | | tattac | |
| cg15239628* | gagtgggtta | 43 | aaaaacaaaa | 44 |
| | ttagggtttt | | actccaataa | |
| | tt | | tctt | |
| cg16035036* | gggttgattt | 45 | cacacaacca | 46 |
| | tattttttgg | | ttcaaaatca | |
| | a | | a | |
| cg16368442* | ggttggtgtg | 47 | aaaaaaacta | 48 |
| | tttgaggg | | cctttcccc | |
| cg17247026* | ttatttattt | 49 | taaccaccca | 50 |
| | tgaggatggt | | caactaaaaa | |
| | tt | | c | |
| cg18328206* | attagtaagt | 51 | ccaaaaatta | 52 |
| | gtgaaggtag | | ttatctcctt | |
| | ggg | | atattc | |
| cg18746831* | gaggtggtga | 53 | aaaacttcat | 54 |
| | gtgaatgtgt | | tcctaaaaac | |
| | tat | | cc | |
| cg19356117* | aggagtgtta | 55 | cctctccaaa | 56 |
| | tgttggaatt | | acaacctata | |
| | tg | | tc | |
| cg19356117 | ggtgatggat | 57 | acctatatcc | 58 |
| | atggaaggat | | ctctatatcc | |
| | t | | ttcc | |
| cg20191310 | aagttaagtt | 59 | ccacaactac | 60 |
| | atagttattt | | taacaaaaca | |
| | ttgttatat | | aatc | |
| cg20458740* | gggtgtttgg | 61 | ccactacaaa | 62 |
| | gtggaaag | | taccacatca | |
| | | | aa | |
| cg20458740 | aagaaagatt | 63 | accataacac | 64 |
| | tagtgggtat | | tcacacctaa | |
| | aagg | | taacc | |
| cg23123895* | tattgtaatt | 65 | ctacaaaaca | 66 |
| | gttttggggt | | atcaaaaccc | |
| | att | | ac | |
| cg24205065* | ggttttagtt | 67 | tacaacaaat | 68 |
| | ttgatattta | | acacacccca | |
| | agaaa | | c | |
| cg24740026 | agaaggaaat | 69 | tcccaacaac | 70 |
| | aggagtggga | | ccccaacaac | |
| | gtt | | | |
| cg24748548* | tgttttgttt | 71 | aacaaaactt | 72 |
| | tgttttgttt | | acaataaacc | |
| | ttt | | aaaat | |
| cg26680097* | ttatggattt | 73 | tttataaacc | 74 |
| | aggtgaggat | | caaattaaaa | |
| | ag | | ac | |
| cg26718232* | tattttgagg | 75 | taataactct | 76 |
| | gggtggagtt | | acccccaaaa | |
| | | | cac | |
| cg27636310* | aagattttgg | 77 | aaaattaaaa | 78 |
| | tttttttttt | | ataccttccc | |
| | tt | | c | |
| CLDN10 | ttaagggata | 79 | cactcccaac | 80 |
| | gggtatgggt | | ccccaaactc | |
| | gt | | | |
| COL11A2 | gtttttgtgt | 81 | aactaaaaat | 82 |
| | gttttgggtt | | aaaatttccc | |
| | a | | ttc | |
| ELFN* | ggatttaggt | 83 | ataactccac | 84 |
| | tatattggga | | taactcctcc | |
| | tgt | | tactc | |
| FAM24A | ttatattaaa | 85 | atatccataa | 86 |
| | tttattttat | | ttcaataaaa | |
| | gtttagg | | ataatat | |
| GALNT9* | gaaaattaaa | 87 | actataaaaa | 88 |
| | gattttagtt | | aactcctaaa | |
| | gttaat | | cttaac | |
| GRIN2A | gtgtgtgtgt | 89 | aaactaaccc | 90 |
| | gagtgtggga | | aacaaccaaa | |
| | g | | aa | |
| HIST1H2BB* | gttattttag | 91 | tttactttaa | 92 |
| | tttgtttgtt | | ctccattttc | |
| | ttttat | | cac | |
| HNRNPF* | ggggaaagtt | 93 | cataatcaaa | 94 |
| | tagagtgtta | | tatacaaacc | |
| | gtta | | aaaata | |
| L0C100192426 | gtttagtagg | 95 | aacaccctct | 96 |
| | tattttagaa | | actctcaact | |
| | ggaag | | actc | |
| MEGF8 | ggggtagttt | 97 | tatacatact | 98 |
| | tttttttatt | | aaaatattcc | |
| | tt | | ataaaacc | |
| MYT1L* | ggaagatatt | 99 | aaaatatcac | 100 |
| | gattgagtat | | tataaccttt | |
| | agagt | | ccc | |
| NAV2* | ggttagggaa | 101 | aaaactctta | 102 |
| | gggaattatt | | aaacaaacct | |
| | | | cc | |
| PTGER | atatagggtt | 103 | accctaaact | 104 |
| | ttgtttgggt | | aaacccaaat | |
| | t | | ac | |
| PTPRN2* | gtttgtttgt | 105 | tcttataaac | 106 |
| | tttatgagag | | ctctcttaaa | |
| | gtta | | tccc | |
| RASSF5 | tttgggttgg | 107 | cctaccttca | 108 |
| | tgtgatttgt | | cacttactaa | |
| | | | tacaac | |
| SLC13A5* | gtttgttttt | 109 | caaactaccc | 110 |
| | aattttgtta | | ctaaaaaact | |
| | tt | | aa | |
| TCERG1L* | atgggtgtta | 111 | cttaaaataa | 112 |
| | aggttaggaa | | ccaacaaccc | |
| | gt | | c | |
| TRH* | tttagaggtg | 113 | caaaaacaaa | 114 |
| | ataggtgtgg | | accactaccc | |
| | a | | | |
| ZMYM2* | tatttttagt | 115 | aaaatcttct | 116 |
| | tgtaatttta | | cttttattcc | |
| | ttaagaa | | tc | |
| ZNF586 | ttgttttgga | 117 | atatcacact | 118 |
| | tatttagttg | | tctttcccaa | |
| | atg | | taa | |

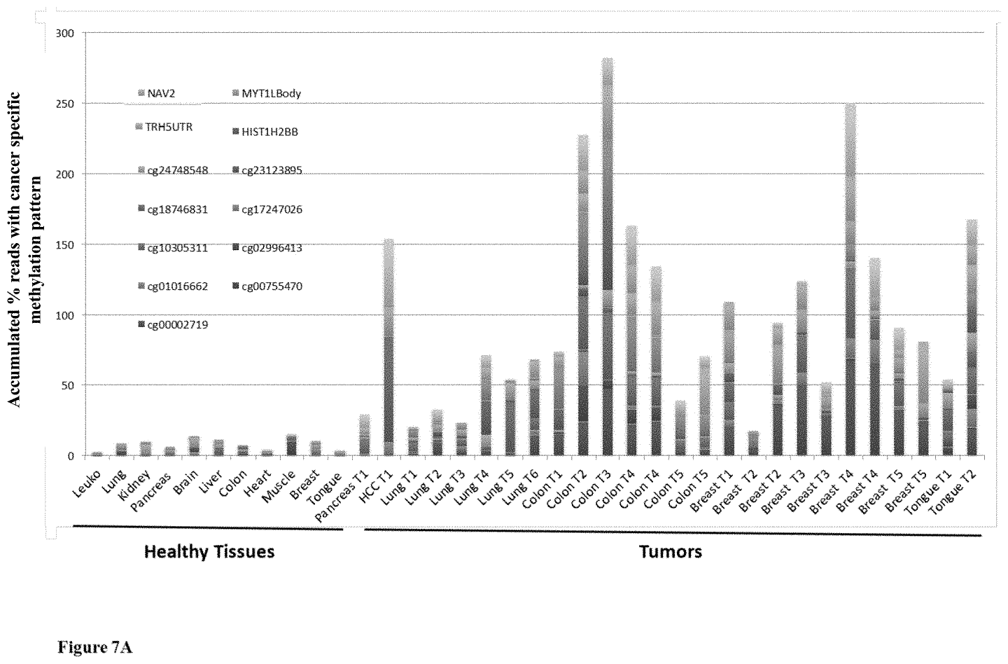

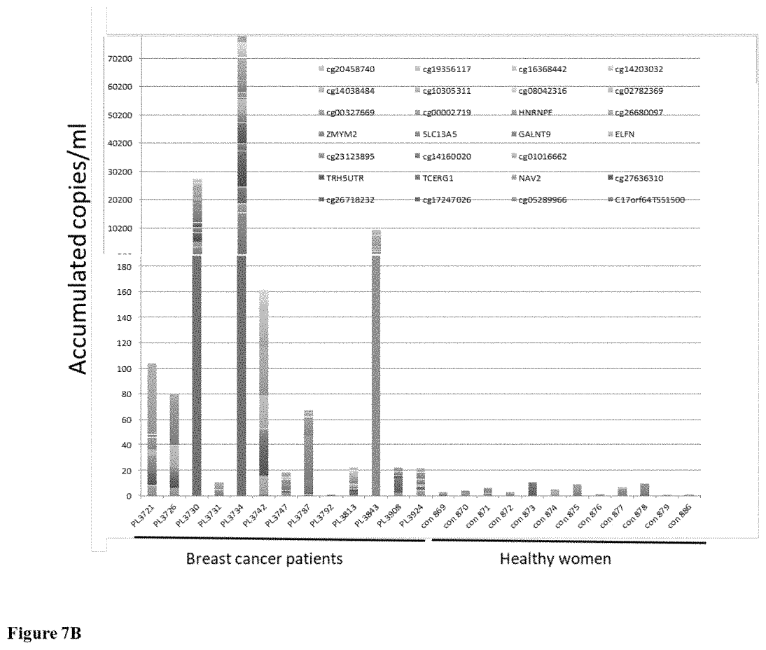

Markers denoted with an * are those shown FIGS. 7A-B. Analysis was with amplification using the primers shown followed by sequencing.

In some embodiments, the region is a genomic region. In some embodiments, the region is a region comprising at least one CpG dinucleotide. It will be understood that as DNA is double stranded, the region comprises both the forward sequence of the region and the reverse complementary sequence of the opposite strand. In some embodiments, the region comprises a plurality of CpG dinucleotides. In some embodiments, the genomic region is a region selected from Table 1. In some embodiments, the region is a region selected from Table 2. In some embodiments, the region is a region reverse complementary to a region selected from Table 1. In some embodiments, the region is a region reverse complementary to a region selected from Table 2. In some embodiments, the region is a region selected from Table 3. In some embodiments, the region is a region reverse complementary to a region selected from Table 3. In some embodiments, the region comprises a central CpG dinucleotide. In some embodiments, the region is from 25, 50, 100, 150, 200, 250, 300, 350, 400, 450, or 500 nucleotides upstream of the central CpG to 25, 50, 100, 150, 200, 250, 300, 350, 400, 450, or 500 nucleotides downstream of the central CpG. Each possibility represents a separate embodiment of the invention. In some embodiments, the region is the central CpG. In some embodiments, the region comprises or consists of from 100 nucleotides upstream to 100 nucleotides downstream of the central CpG. In some embodiments, the region is from 100 nucleotides upstream to 100 nucleotides downstream of the central CpG. In some embodiments, the region is 201 nucleotides in size. In some embodiments, the region comprises or consists of from 250 nucleotides upstream to 250 nucleotides downstream of the central CpG. In some embodiments, the region is from 250 nucleotides upstream to 250 nucleotides downstream of the central CpG. In some embodiments, the region is 501 nucleotides in size. In some embodiments, the region comprises at least 1, 2, 3, 4, 5, 6, 7, 8, 9 or 10 CpG dinucleotides. Each possibility represents a separate embodiment of the invention. In some embodiments, the region comprises at least 1, 2, 3, 5, 10, 15, 20, 25, 50, 100, 150, 200, 250, 300, 350, 400, 450, or 500 nucleotides. Each possibility represents a separate embodiment of the invention. In some embodiments, the region comprises at most 1, 2, 3, 5, 10, 15, 20, 25, 50, 100, 150, 200, 250, 300, 350, 400, 450, 500, 600 700, 750, 800, 900, or 1000 nucleotides. Each possibility represents a separate embodiment of the invention.

In some embodiments, the region is a region methylated in cancer and is selected from Table 1. In some embodiments, methylated is hypermethylated. In some embodiments, hypermethylated is as compared to a non-cancerous tissue or cell type. In some embodiments, hypermethylated is as compared to a healthy tissue or cell type. In some embodiments, hypermethylated is as compared to cfDNA from healthy subjects. In some embodiments, the region is a region from Table 1 and the cancer-specific methylation pattern is methylation in the region. In some embodiments, the region is a region from Table 1 and the cancer-specific methylation pattern is methylation of the central CpG. In some embodiments, methylation in the region is methylation of at least 1, 2, 3, 4, 5, 6, 7, 8, 9 or 10 CpG dinucleotides. Each possibility represents a separate embodiment of the invention. In some embodiments, methylation in the region is methylation of at least 5 CpG dinucleotides. In some embodiments, methylation in the region is methylation of at least 8 CpG dinucleotides. In some embodiments, methylation in the region is methylation of the central CpG and at least 1, 2, 3, 4, 5, 6, 7, 8, 9 or 10 other CpG dinucleotides in the region. Each possibility represents a separate embodiment of the invention. In some embodiments, methylation of the region is methylation of the central CpG and at least one other CpG in the region. In some embodiments, methylation of the region is methylation of the central CpG and at least four other CpGs in the region. In some embodiments, methylation of the region is methylation of the central CpG and at least seven other CpGs in the region.

In some embodiments, the region is a region unmethylated in cancer and is selected from Table 2. In some embodiments, unmethylated in hypomethylated. In some embodiments, hypomethylated is as compared to a non-cancerous tissue or cell type. In some embodiments, hypomethylated is as compared to a healthy tissue or cell type. In some embodiments, hypomethylated is as compared to cfDNA from healthy subjects. In some embodiments, the region is a region from Table 2 and the cancer-specific methylation pattern is unmethylation in the region. In some embodiments, the region is a region from Table 2 and the cancer-specific methylation pattern is unmethylation of the central CpG. In some embodiments, unmethylation in the region is unmethylation of at least 1, 2, 3, 4, 5, 6, 7, 8, 9 or 10 CpG dinucleotides. Each possibility represents a separate embodiment of the invention. In some embodiments, unmethylation in the region is unmethylation of at least 5 CpG dinucleotides. In some embodiments, unmethylation in the region is unmethylation of at least 8 CpG dinucleotides. In some embodiments, unmethylation in the region is unmethylation of the central CpG and at least 1, 2, 3, 4, 5, 6, 7, 8, 9 or 10 other CpG dinucleotides in the region. Each possibility represents a separate embodiment of the invention. In some embodiments, unmethylation of the region is unmethylation of the central CpG and at least one other CpG in the region. In some embodiments, unmethylation of the region is unmethylation of the central CpG and at least four other CpGs in the region. In some embodiments, unmethylation of the region is unmethylation of the central CpG and at least seven other CpGs in the region.

In some embodiments, at least one region is 1, 2, 3, 4, 5, 10, 15, 20, 25, 30, 35, 40, 45, 50, 60, 65, 70, 75, 80, 85 or 87 regions. Each possibility represents a separate embodiment of the invention. In some embodiments, at least one region is at least 1, 2, 3, 4, 5, 10, 15, 20, 25, 30, 35, 40, 45, 50, 60, 65, 70, 75, 80, 85 or 87 regions. Each possibility represents a separate embodiment of the invention. In some embodiments, at least one region is at least 20 regions. In some embodiments, at least one region is at least 13 regions. In some embodiments, at least one region is at least 27 regions. It will be understood by a skilled artisan that the more regions are examined the more reliable is a negative result; however, a positive result from even one region is an indication of cancer. Using more regions also increases the reliability of a positive result. Thus, use of more regions, will increase sensitivity and specificity. A skilled artisan will also appreciate that regions from Table 1 and Table 2 can be combined during examination, but each will be judged by its specific cancer-specific pattern.

In some embodiments, the at least one region is selected from the regions examined in FIG. 7A. In some embodiments, the at least one region is selected from the regions examine in FIG. 7B. In some embodiments, the at least one region is the regions examined in FIG. 7A. In some embodiments, the at least one region is the regions examined in FIG. 7B.

As used herein, the term “cancer specific methylation pattern” and “cancer specific pattern” are used synonymously and interchangeably and refer to the methylation or lack of methylation on at least one CpG dinucleotide that if differential between healthy tissue and at least one cancer. A methylation pattern can be at a single CpG, i.e. the central CpG or can be over an entire region, or over several CpGs of a region. In some embodiments, cancer specific pattern is methylation in cancer and unmethylation in healthy tissue. In some embodiments, the healthy tissue is matched to the cancer. In some embodiments, the matched tissue is from the same cell type or tissue as the cancer. In some embodiments, cancer specific pattern is methylation in cancer and unmethylation in healthy leukocytes. In some embodiments, cancer specific pattern is methylation in cancer and unmethylation in cfDNA from healthy subjects. In some embodiments, cancer specific pattern is unmethylation in cancer and methylation in healthy tissue. In some embodiments, cancer specific pattern is unmethylation in cancer and methylation in healthy leukocytes. In some embodiments, cancer specific pattern is unmethylation in cancer and methylation in cfDNA from healthy subjects.

In some embodiments, the cancer specific methylation pattern is a pan-cancer pattern. In some embodiments, the cancer pattern is for at least 1, 2, 3, 4, 5, 6, 7, 8, 9, or 10 cancers. Each possibility represents a separate embodiment of the invention. In some embodiments, the cancer pattern is for at least 1 cancer. In some embodiments, the cancer pattern is for 1 cancer. In some embodiments, the cancer pattern is for a plurality of cancers. In some embodiments, the cancer pattern is for a specific pattern. In some embodiments, the pattern for specific cancers is based on methylation levels provided in Table 3. In some embodiments, the pattern for specific cancers is selected from Table 3. In some embodiments, the pattern for specific cancers is based on specific regions for the specific cancer. In some embodiments, the cancer and the region are matched based on methylation levels provided in Table 3. In some embodiments, the cancer and region are matched based on differential methylation from healthy tissue based on methylation levels in Table 3. In some embodiments, the cancer and region are matched based on differential methylation from healthy cfDNA samples based on methylation levels in Table 3.

According to another aspect, there is provided an array, comprising at least 1 methylation specific oligonucleotide, wherein the methylation specific oligonucleotide comprises a sequence reverse complementary to a sequence of a genomic region provide in Table 1 or Table 2.

In some embodiments, the array is an array of oligonucleotides. In some embodiments, the oligonucleotides are in solution. In some embodiments, the oligonucleotides are immobilized to a solid support. In some embodiments, the array further comprises a solid support. In some embodiments, the oligonucleotides are pooled. In some embodiments, each oligonucleotide is in a separate container. In some embodiments, the oligonucleotide is immobilized to one support. In some embodiments, the solid support is a chip. In some embodiments, the oligonucleotides are each immobilized to a separate solid support. In some embodiments, the solid support is a bead. In some embodiments, each oligonucleotide is immobilized to a bead. In some embodiments, each bead comprises a plurality of oligonucleotides immobilized thereto. In some embodiments, the oligonucleotides immobilized to a bead are all the same oligonucleotide. In some embodiments, a plurality of oligonucleotides is immobilized to a plurality of solid supports. In some embodiments, the bead is a magnetic bead. In some embodiments, the bead is a paramagnetic bead. In some embodiments, the bead is configured for isolation. In some embodiments, the oligonucleotide is conjugated to a capture moiety. In some embodiments, the capture moiety is the bead. As used herein, a capture moiety is a molecule that can be isolated by binding to a capturing molecule. For example, the oligonucleotide can be conjugated to biotin (capture moiety) and then captured by a streptavidin column (the capturing molecule). Any capturing system may be used so that the oligonucleotides can be isolated after binding to target DNA.

In some embodiments, the oligonucleotide is connected to the solid support by a linker. In some embodiments, the linker is a nucleic acid linker. In some embodiments, the linker is a flexible linker. In some embodiments, the liker is a bond. In some embodiments, the bond is a reversible bond. In some embodiments, the bond is a covalent bond. In some embodiments, the linker is a cleavable linker.

In some embodiments, a reverse complementary sequence is a sequence that hybridizes to the genomic region. In some embodiments, the array comprises at least 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 87, 100, 150, 200, 250, 300, 350, 400, 550, 500, 600, 700, 800, 900, or 1000 oligonucleotides. Each possibility represents a separate embodiment of the invention. In some embodiments, the array comprises at least 10 oligonucleotides. In some embodiments, the array comprises at least 13 oligonucleotides. In some embodiments, the array comprises at least 20 oligonucleotides. In some embodiments, the array comprises at least 27 oligonucleotides. In some embodiments, the array comprises oligonucleotides that are reverse complementary to at least 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85 or 87 regions. Each possibility represents a separate embodiment of the invention. In some embodiments, the array comprises oligonucleotides reverse complementary to at least 10 regions. In some embodiments, the array comprises oligonucleotides reverse complementary to at least 13 regions. In some embodiments, the array comprises oligonucleotides reverse complementary to at least 20 regions. In some embodiments, the array comprises oligonucleotides reverse complementary to at least 27 regions. In some embodiments, more than one oligonucleotide is reverse complementary to a region. In some embodiments, a plurality of oligonucleotides binds to one region. In some embodiments, the array comprises a plurality of oligonucleotides that cover at least 2 CpGs in a region. In some embodiments, the array comprises oligonucleotides that cover all of a region. In some embodiments, the array comprises oligonucleotides that cover all CpGs in a region. In some embodiments, the array comprises oligonucleotides that cover all differentially methylated CpGs in a region. In some embodiments, the array comprises at least one methylation specific oligonucleotide reverse complementary to each region from Table 1. In some embodiments, the array comprises at least one methylation specific oligonucleotide reverse complementary to each region from Table 2. In some embodiments, the array comprises at least one methylation specific oligonucleotide reverse complementary to each region from Table 1 and Table 2.

In some embodiments, the array comprises an oligonucleotide that binds a region when the region is methylated. In some embodiments, the array comprises an oligonucleotide that binds a region when the region is unmethylated. In some embodiments, the oligonucleotides bind the region after bisulfite conversion. In some embodiments, the oligonucleotide binds to a sequence of the region after bisulfite conversion. In some embodiments, an oligonucleotide binds to a methylated region after bisulfite conversion. In some embodiments, an oligonucleotide binds to an unmethylated region after bisulfite conversion. It will be understood that the sequence of a region may change after bisulfite conversion is a CpG is unmethylated and thus an oligonucleotide may be reverse complementary only before or only after bisulfite conversion or may be reverse complementary to both. In some embodiments, the array comprises a first oligonucleotide that binds a region when the region is methylated and a second oligonucleotide that binds the region when the region is unmethylated.

In some embodiments, a methylation specific oligonucleotide is a methylation specific primer. In some embodiments, the oligonucleotide is a primer. In some embodiments, the methylation specific oligonucleotide hybridizes in the presence of methylation. In some embodiments, the methylation specific oligonucleotide only hybridizes in the presence of methylation. In some embodiments, the methylation specific oligonucleotide hybridizes in the absence of methylation. In some embodiments, the methylation specific oligonucleotide only hybridizes in the absence of methylation. In some embodiments, the methylation specific oligonucleotide is reverse complementary to a sequence of a region from Table 1 and is not complementary to sequence of a region from Table 1 wherein a cytosine of a CpG dinucleotide is converted to a thymine. In some embodiments, the methylation specific oligonucleotide is reverse complementary to a sequence of a region from Table 1 wherein a cytosine of a CpG dinucleotide is converted to a thymine and is not complementary to sequence of a region from Table 1. In some embodiments, the methylation specific oligonucleotide is reverse complementary to a sequence of a region from Table 2 and is not complementary to sequence of a region from Table 2 wherein a cytosine of a CpG dinucleotide is converted to a thymine. In some embodiments, the methylation specific oligonucleotide is reverse complementary to a sequence of a region from Table 2 wherein a cytosine of a CpG dinucleotide is converted to a thymine and is not complementary to sequence of a region from Table 2. In some embodiments, a plurality of oligonucleotides comprises the full sequence of a region. In some embodiments, the oligonucleotides are tiled to cover an entire region. It will be understood by a skilled artisan that if a region is 501 nucleotides, and a single oligonucleotide is, for example 130 nucleotides. Then, at least 4 oligonucleotides would be required to cover the entire region. Further, if the oligonucleotides contained some overlap then even more oligonucleotides might be required to cover the entire 501 nucleotides. Overlap creates redundancy that may increase the sensitivity of the array. If 130 nucleotide oligonucleotides are used, with 30 nucleotides of overlap, then 5 oligonucleotides would cover an entire 501 nucleotide region.

In some embodiments, the oligonucleotide is specific to its target. In some embodiments, the target is a target sequence. In some embodiments, the target is a target region. In some embodiments, the target is a target gene. In some embodiments, the oligonucleotide specifically binds in the genomic region. In some embodiments, the oligonucleotide specifically hybridizes to the genomic region. In some embodiments, the oligonucleotide does not hybridize to a sequence outside of the genomic region. In some embodiments, the oligonucleotide does not cause off target effects. In some embodiments, the oligonucleotide uniquely hybridizes to the target region. It will be understood by a skilled artisan that the oligonucleotide allows for identification and/or isolation of the genomic regions of Tables 1 and 2. Thus, an oligonucleotide that hybridizes elsewhere or mis-hybridizes elsewhere is suboptimal. In some embodiments, the oligonucleotide is 100% reverse complementary to its target. In some embodiments, the oligonucleotide is at least 85, 90, 92, 94, 95, 97, 99, or 100% reverse complementary to its target. Each possibility represents a separate embodiment of the invention. In some embodiments, the oligonucleotide is at most 60, 65, 70, 75, 80, 85, 90, 92, 94, 95, 97, or 99% reverse complementary to a sequence outside of the genomic region. Each possibility represents a separate embodiment of the invention. In some embodiments, the oligonucleotide is at most 80% reverse complementary to a sequence outside of the genomic region. In some embodiments, the oligonucleotide is at most 85% reverse complementary to a sequence outside of the genomic region. In some embodiments, the oligonucleotide is at most 90% reverse complementary to a sequence outside of the genomic region.

In some embodiments, the oligonucleotide is reverse complementary to a region. In some embodiments, the oligonucleotide is reverse complementary to a genomic region. In some embodiments, the oligonucleotide is homologous to the region. In some embodiments, the oligonucleotide is reverse complementary to the opposite strand of the region. In some embodiments, the oligonucleotide is reverse complementary to a region comprises a central CpG. In some embodiments, the oligonucleotide is reverse complementary to a region within 100 nucleotides upstream and downstream of a central CpG. In some embodiments, the oligonucleotide is reverse complementary to a region within 500 nucleotides upstream and downstream of a central CpG.

In some embodiments, the oligonucleotide comprises at least 8, 10, 12, 14, 16, 18, 20, 22, 24, 26, 28, 30, 40, 50, 60, 70, 80, 90, 100, 110, 120, 130, 140, 150, 160, 170, 180, 190, 200, 210, 220, 230, 240 or 250 nucleotides. Each possibility represents a separate embodiment of the invention. In some embodiments, the oligonucleotide comprises at least 50 nucleotides. In some embodiments, the oligonucleotide comprises at least 75 nucleotides. In some embodiments, the oligonucleotide comprises at least 100 nucleotides. In some embodiments, the oligonucleotide comprises at least 120 nucleotides. In some embodiments, the oligonucleotide comprises at least 130 nucleotides. In some embodiments, the oligonucleotide comprises at least 150 nucleotides. In some embodiments, the oligonucleotide is about the size of DNA wrapped around one nucleosome. In some embodiments, the oligonucleotide comprises at most 14, 16, 18, 20, 22, 24, 26, 28, 30, 32, 34, 36, 38, 40, 42, 44, 46, 48, 50, 60, 70, 80, 90, 100, 110, 120, 130, 140, 150, 160, 170, 180, 190, 200, 210, 220, 230, 240 or 250 nucleotides. Each possibility represents a separate embodiment of the invention. In some embodiments, the oligonucleotide comprises between 8-40, 8-35, 8-30, 8-25, 8-20, 10-40, 10-35, 10-30, 10-25, 10-20, 12-40, 12-35, 12-30, 12-25, 12-20, 14-40, 14-35, 14-30, 14-25, 14-20, 15-40, 15-35, 15-30, 15-25, 15-20, 16-40, 16-35, 16-30, 16-25, 16-20, 18-40, 18-35, 18-30, 18-25, 18-20, 20-40, 20-35, 20-30, 20-25, 50-200, 50-150, 50-140, 50-130, 50-120, 50-110, 50-100, 60-200, 60-150, 60-140, 60-130, 60-120, 60-110, 60-100, 70-200, 70-150, 70-140, 70-130, 70-120, 70-110, 70-100, 80-200, 80-150, 80-140, 80-130, 80-120, 80-110, 80-100 90-200, 90-150, 90-140, 90-130, 90-120, 90-110, 90-100, 100-200, 100-150, 100-140, 100-130, 100-120, 100-110, 110-200, 110-150, 110-140, 110-130, 110-120, 120-200, 120-150, 120-140, 120-130, 130-200, 130-150, 130-140, 140-200, 140-150, or 150-200. Each possibility represents a separate embodiment of the invention. In some embodiments, an oligonucleotide is homologous to the region and is devoid of cytosines. In some embodiments, an oligonucleotide is reverse complementary to the region and is devoid of cytosines. In some embodiments, an oligonucleotide is homologous to the region and is devoid of guanines. In some embodiments, an oligonucleotide is reverse complementary to the region and is devoid of guanines.

In some embodiments, the oligonucleotide comprises a sequence for amplification. In some embodiments, each oligonucleotide of the array comprises a universal sequence. In some embodiments, a plurality of oligonucleotides of the array comprises a universal sequence. In some embodiments, an oligonucleotide comprises a universal sequence. In some embodiments, the universal sequence is 5′ to the reverse complementary sequence. In some embodiments, the universal sequence is a sequence of a forward primer. In some embodiments, the oligonucleotide comprises a nucleotide barcode. In some embodiments, the oligonucleotide comprises a unique molecular identifier (UMI). In some embodiments, the oligonucleotide comprises a region homologous to or reverse complementary to a sequencing primer. In some embodiments, the universal sequence comprises the region homologous or reverse complementary to a sequencing primer. In some embodiment, the region homologous or verse complementary to a sequencing primer is 5′ to a region for amplification. A skilled artisan will appreciate that after binding a genomic region with a cancer-specific methylation, it may be beneficial to sequence the region. Sequencing is well known in the art, but generally requires amplification as a first step. This amplification is often clonal and can be performed on the solid support (i.e. bead) or off it. The clonally amplified copies are then sequenced, and the region where the sequencing primer binds can be on the oligonucleotide of added at the other end of the amplification product. In some embodiments, an adapter is added to the target DNA molecule. The adapter can also have the region homologous or reverse complementary to the sequencing primer.

According to another aspect, there is provided a kit comprising an array of the invention and a nucleic acid adapter.

In some embodiments, the nucleic acid adapter is a double stranded adapter. In some embodiments, the nucleic acid adapter is a single stranded adapter. In some embodiments, the adapter is configured to be ligated to a target molecule. In some embodiments, the adapter is a blunt end adapter. In some embodiments, the adapter comprises an overhang. In some embodiments, the overhang is a T/A overhang. In some embodiments, the T/A overhang is a T overhang. In some embodiments, the T/A overhang is an A overhang. It will be understood that many polymerases used for reverse transcription leave an A overhang. Thus, the adapter may have a T/A overhang to facilitate T/A overhang ligation of the adapter after the reverse transcription. In some embodiments, the target molecule is a DNA. In some embodiments, the DNA is bisulfite converted DNA. In some embodiments, the adapter is a DNA adapter. In some embodiments, the adapter is an RNA adapter. In some embodiments, the adapter is a DNA, RNA, LNA or PNA adapter. In some embodiments, the adapter comprises a sequence for amplification. In some embodiments, the sequence if for amplification of the target molecule. In some embodiments, the amplification is for after capture of the target molecule to an oligonucleotide of the array. In some embodiments, the adapter comprises a reverse primer. In some embodiments, the adapter comprises a region homologous or reverse complementary to a sequencing primer. In some embodiments, the kit further comprises a ligase. In some embodiments, the ligase is a double stranded ligase. In some embodiments, the ligase is a single stranded ligase. In some embodiments, the ligase is a blunt end ligase. In some embodiments, the ligase is an overhang ligase. In some embodiments, the overhang is a T/A overhang.

In some embodiments, the kit further comprises a reagent for amplification. In some embodiment, the reagent is a polymerase. In some embodiments, the polymerase produces a free A overhang at the end of a synthesized strand. In some embodiments, the reagent is a free nucleotide. In some embodiments, the free nucleotide is all four DNA oligonucleotides. In some embodiments, the free nucleotide is a pool of free nucleotides. In some embodiments, the reagent is a primer. In some embodiments, the kit further comprises a primer. In some embodiments, the primer is for amplification of a target molecule hybridized to an oligonucleotide of the array. In some embodiments, the primer is a forward primer in some embodiments, the primer is a reverse primer. In some embodiments, the kit comprises a forward and a reverse primer. In some embodiments, the kit comprises reagents sufficient for amplification of a target molecule hybridized to the array.

As used herein, the term “about” when combined with a value refers to plus and minus 10% of the reference value. For example, a length of about 1000 nanometers (nm) refers to a length of 1000 nm+−100 nm.

It is noted that as used herein and in the appended claims, the singular forms “a,” “an,” and “the” include plural referents unless the context clearly dictates otherwise. Thus, for example, reference to “a polynucleotide” includes a plurality of such polynucleotides and reference to “the polypeptide” includes reference to one or more polypeptides and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

In those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

Various embodiments and aspects of the present invention as delineated hereinabove and as claimed in the claims section below find experimental support in the following examples.

### EXAMPLES

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Strategies for Protein Purification and Characterization—A Laboratory Course Manual” CSHL Press (1996); all of which are incorporated by reference. Other general references are provided throughout this document.

### Example 1: Superiority of Using Multiple Sites

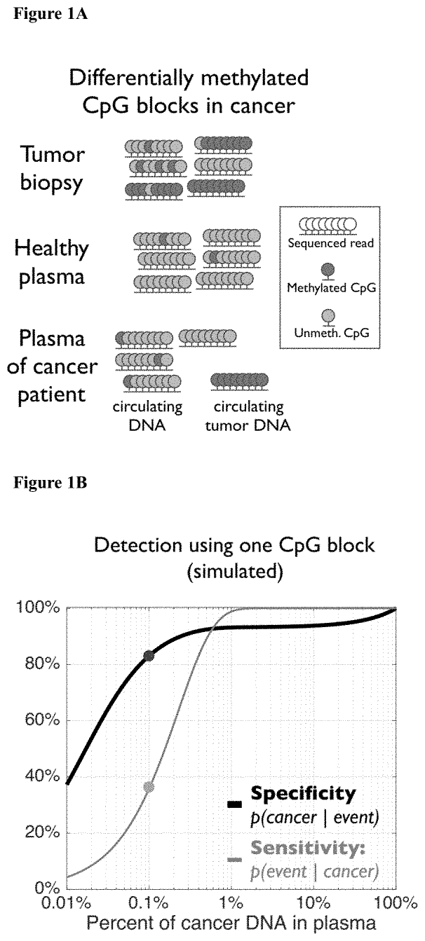

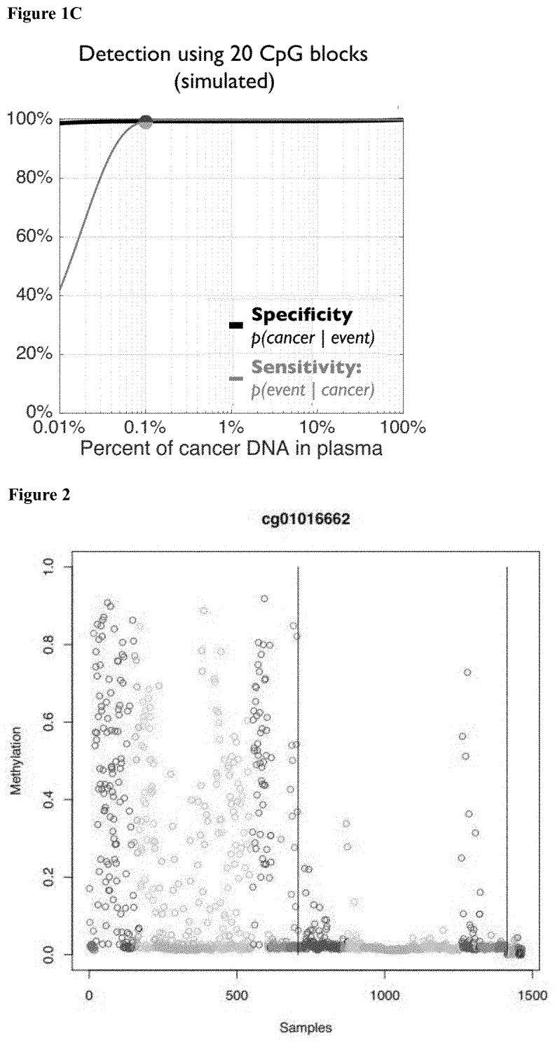

A useful cancer marker is one that is differentially methylated as compared to healthy tissue, and specifically the same tissue type as the one from which the cancer originated. Further, for the purposes of a liquid biopsy, since most of the cfDNA in blood is from blood cells it is also beneficially is the marker is differentially methylated as compared to healthy leukocytes. Generally, these differentially methylated regions are methylated in cancer cells—often across multiple cancer types—but are ubiquitously (or nearly ubiquitously) unmethylated in all healthy cell type (FIG. 1A). The inverse is also possible, where the region is unmethylated in cancer cells, but methylated in healthy cells.

Using statistical simulations, the aggregated statistical power of neighboring CpGs in multiple genomic regions was estimated (FIG. 1B). While a single region with 5 CpGs might not suffice for the detection of circulating tumor DNA in a sensitive and specific manner—at a concentration of 0.1% tumor DNA in the plasma, only 38% of cancer patients are expected to present this biomarker (sensitivity), and its presence is not limited to cancer patients (specificity of 83%; FIG. 1B)—a combination of 20 such regions yield sensitivity and specificity of ≥99% (FIG. 1C).

### Example 2: Whole Genome Analysis

In order to determine differentially methylated regions whole genome bisulfite conversion analysis was performed. Genomic regions were selected by one of two criteria: (1) unmethylated in leukocytes (<10%), in healthy biopsies (<10%), but are methylated (>50%) in at least one cancer types; or (2) unmethylated in leukocytes (<10%), in healthy biopsies (<30%), but are methylated (>50%) in at least two cancer types. Also selected are regions with the converse patterns: (1) methylated in leukocytes (>90%), in healthy biopsies (>90%), but are unmethylated (<50%) in at least one cancer types; or (2) methylated in leukocytes (>90%), in healthy biopsies (>70%), but are unmethylated (<50%) in at least two cancer types.

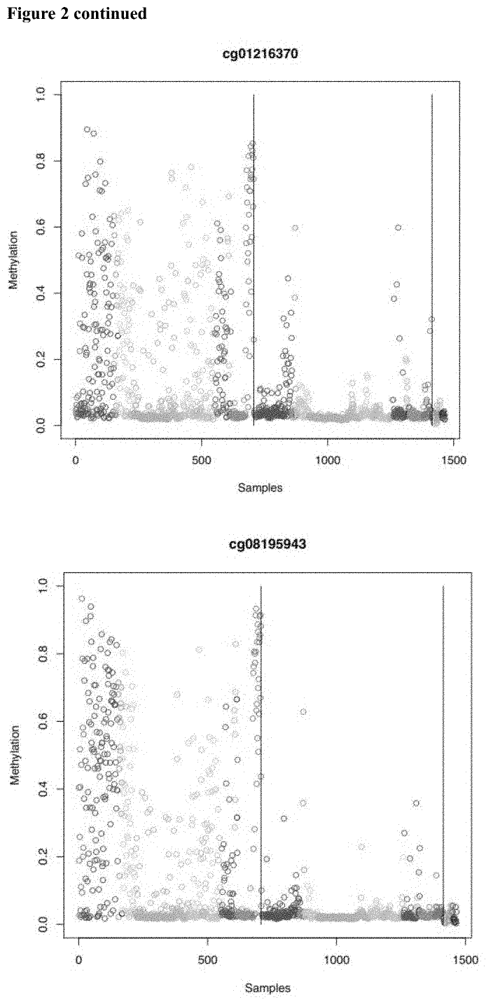

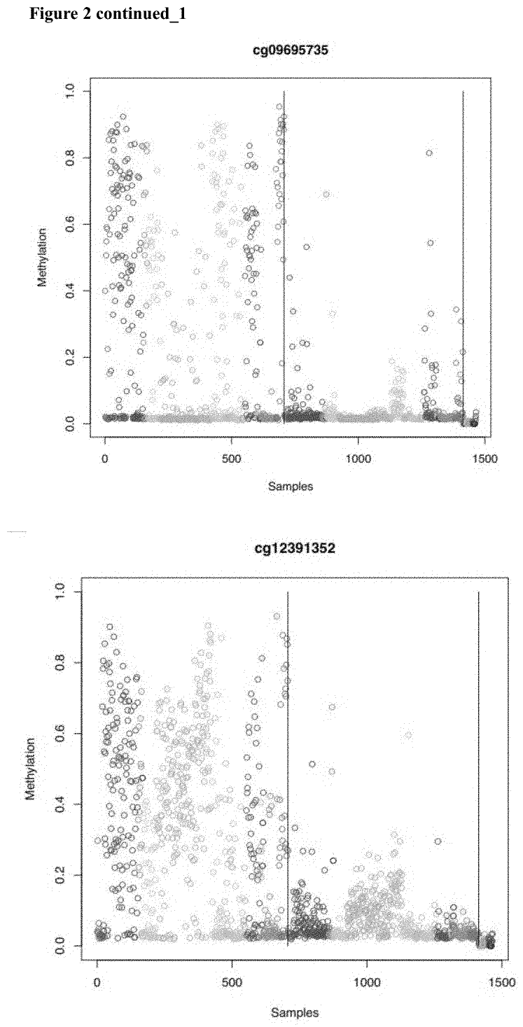

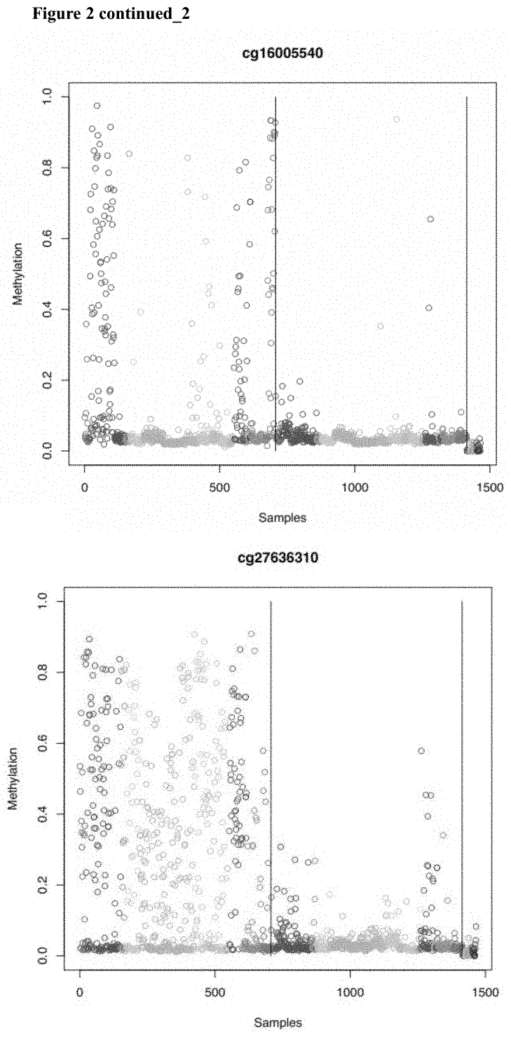

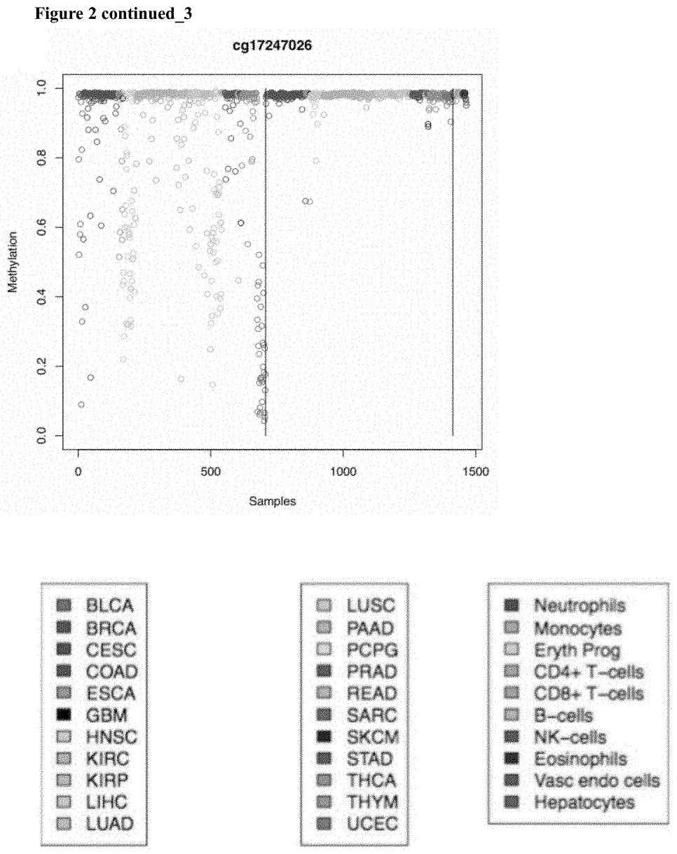

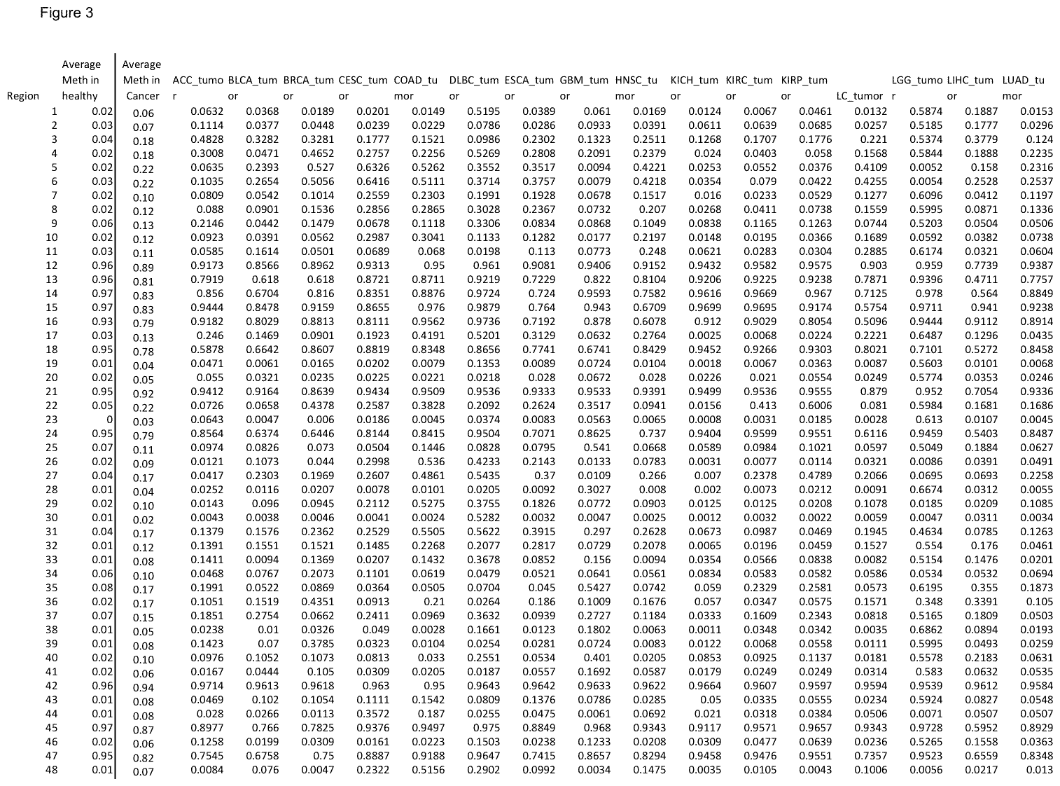

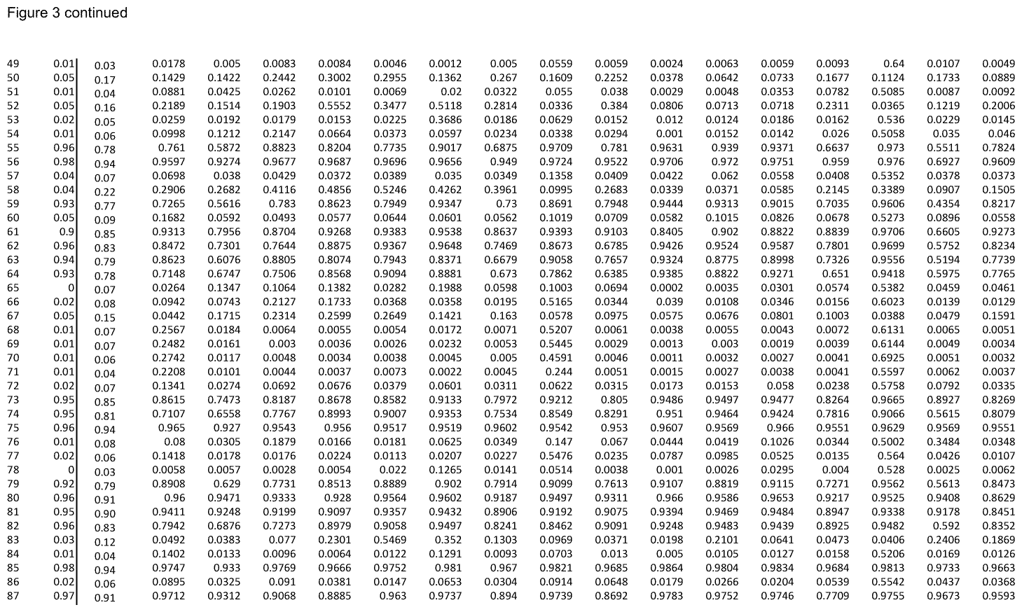

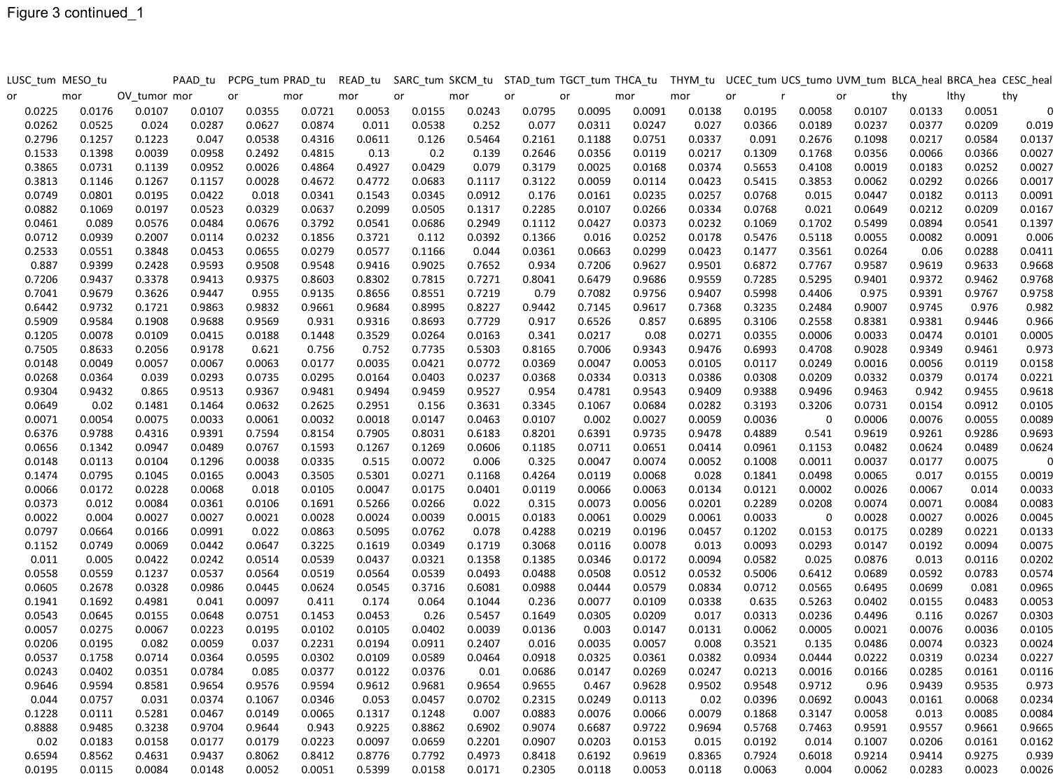

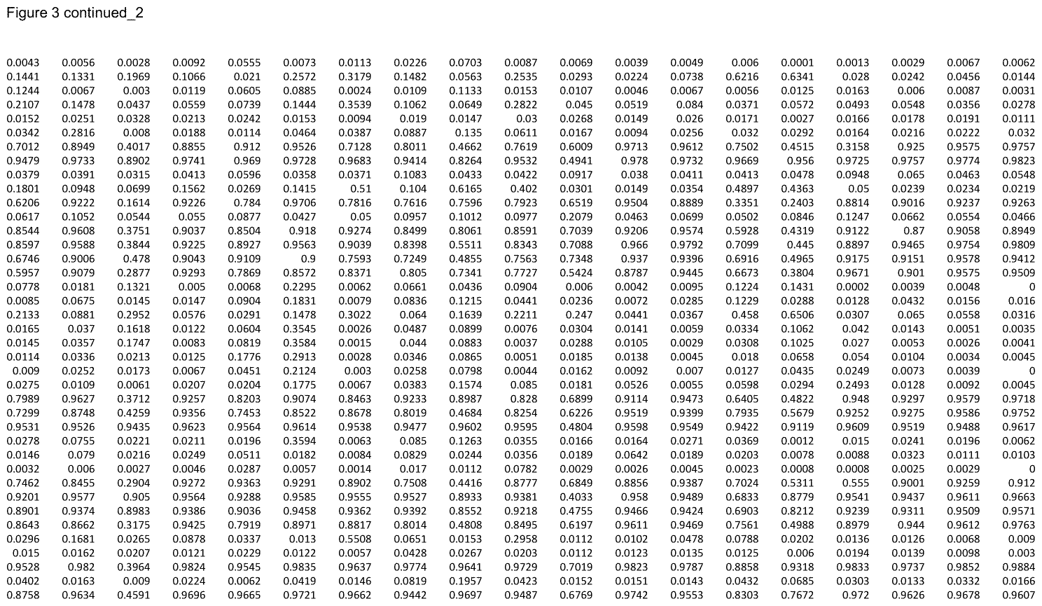

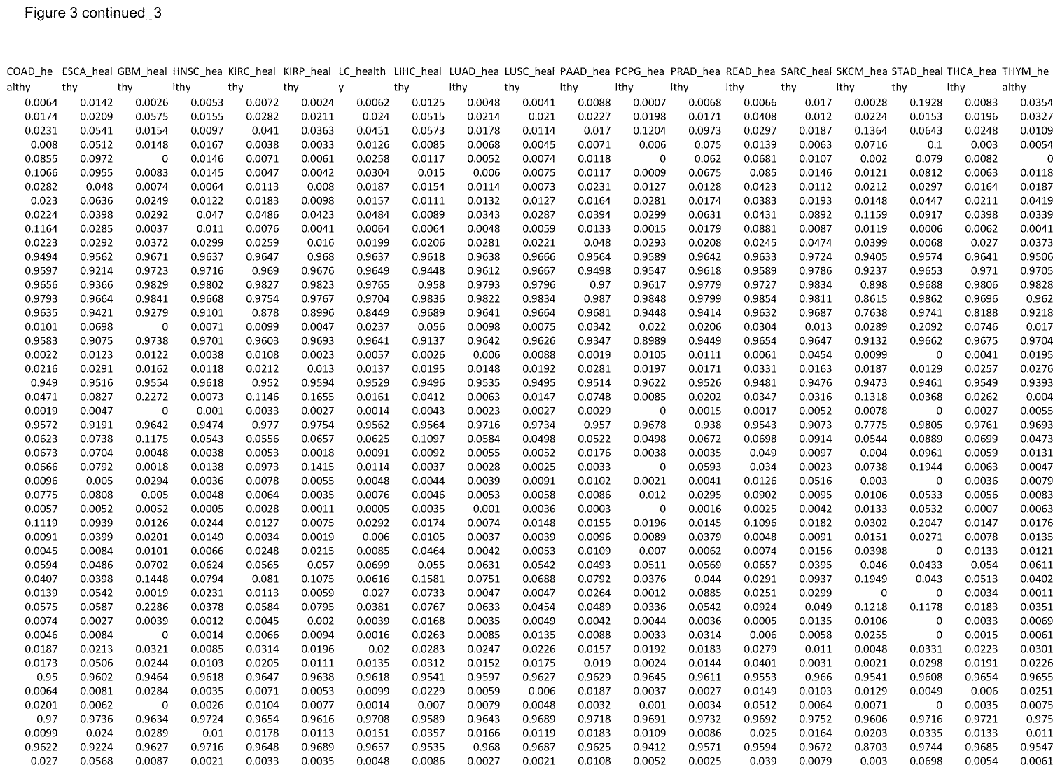

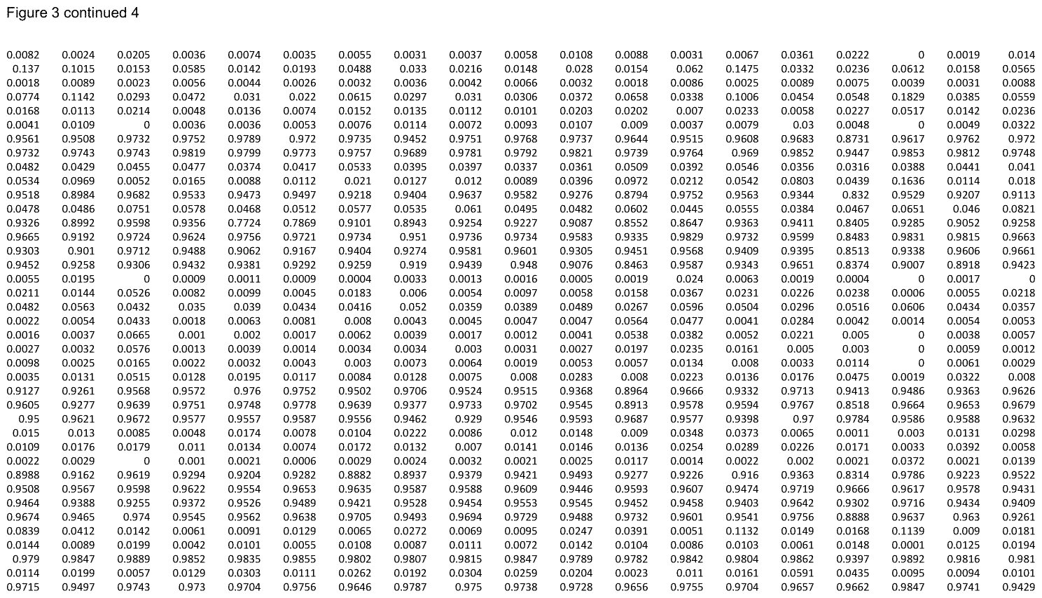

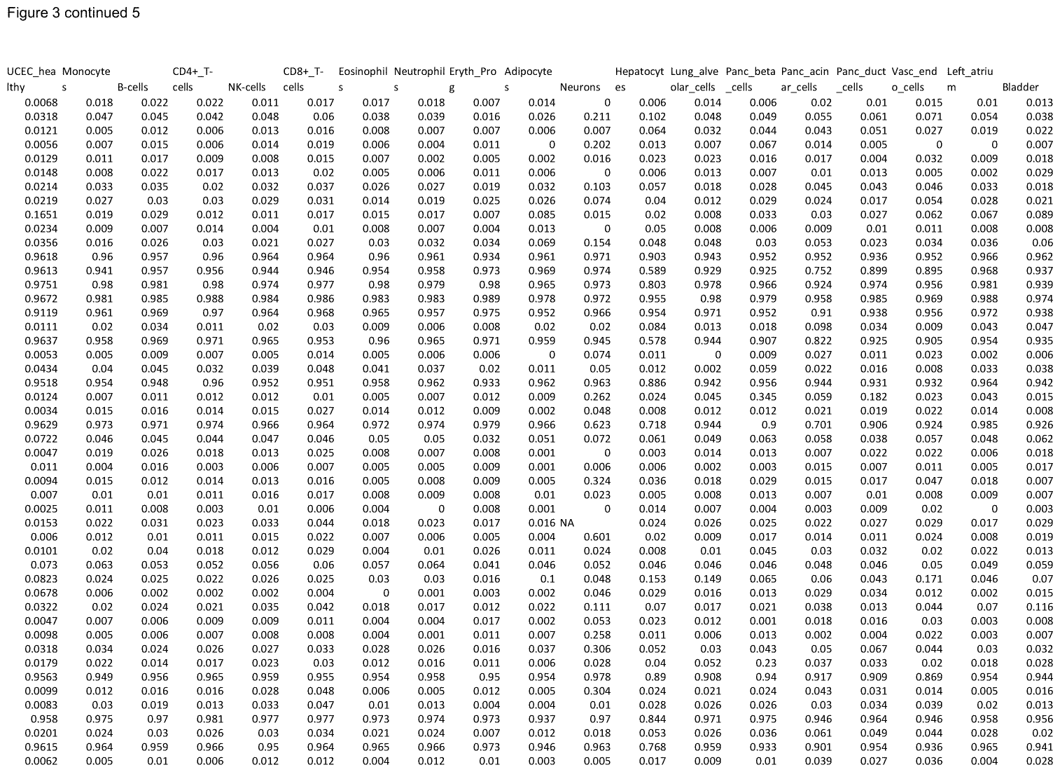

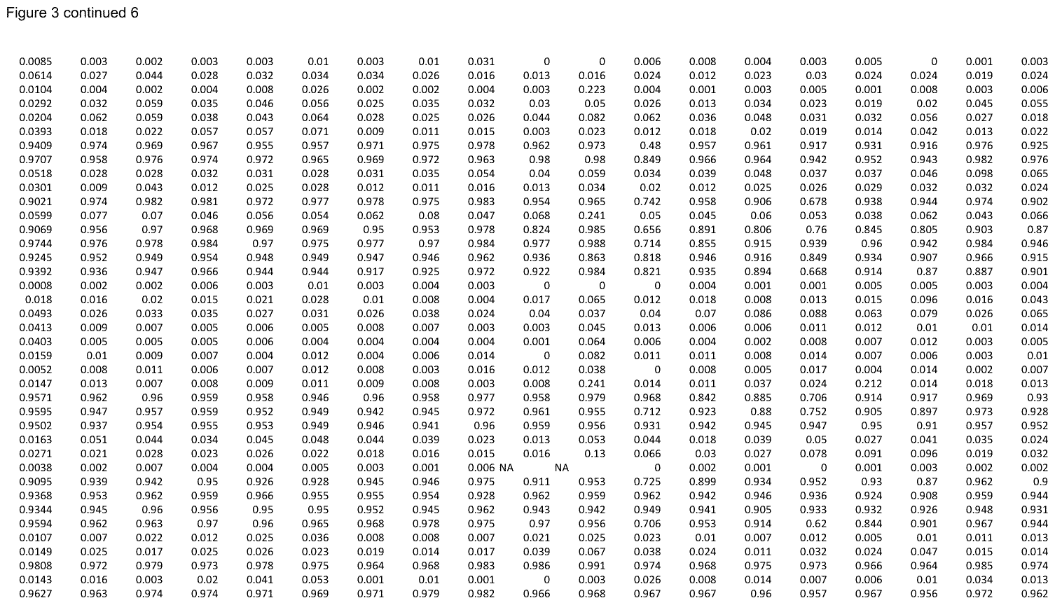

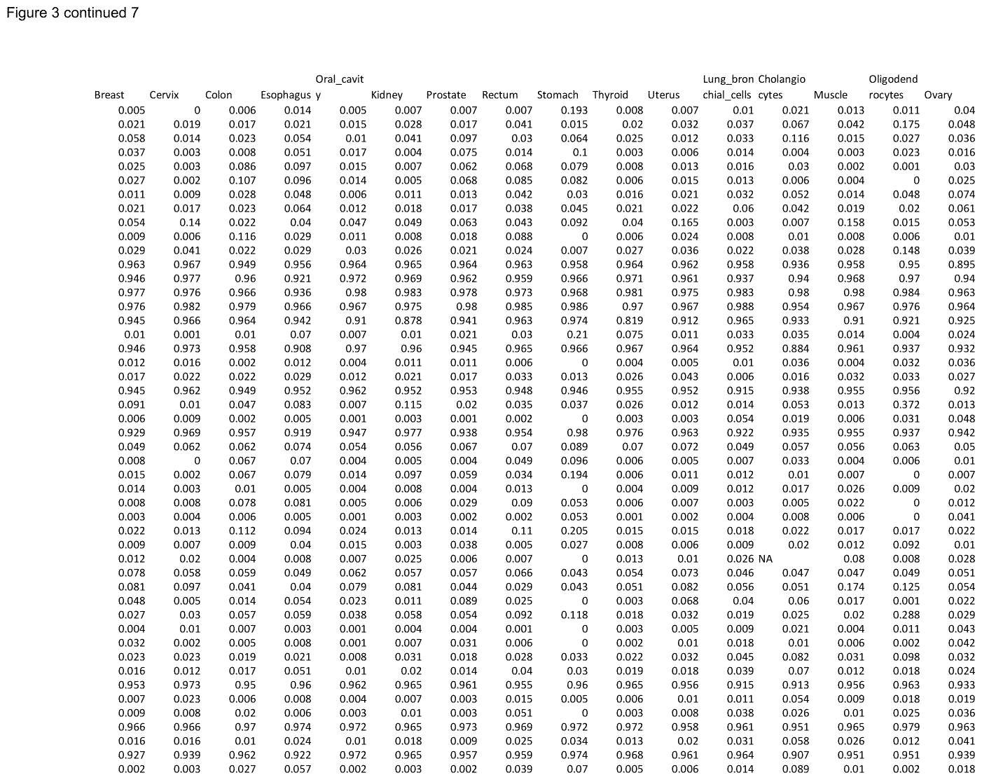

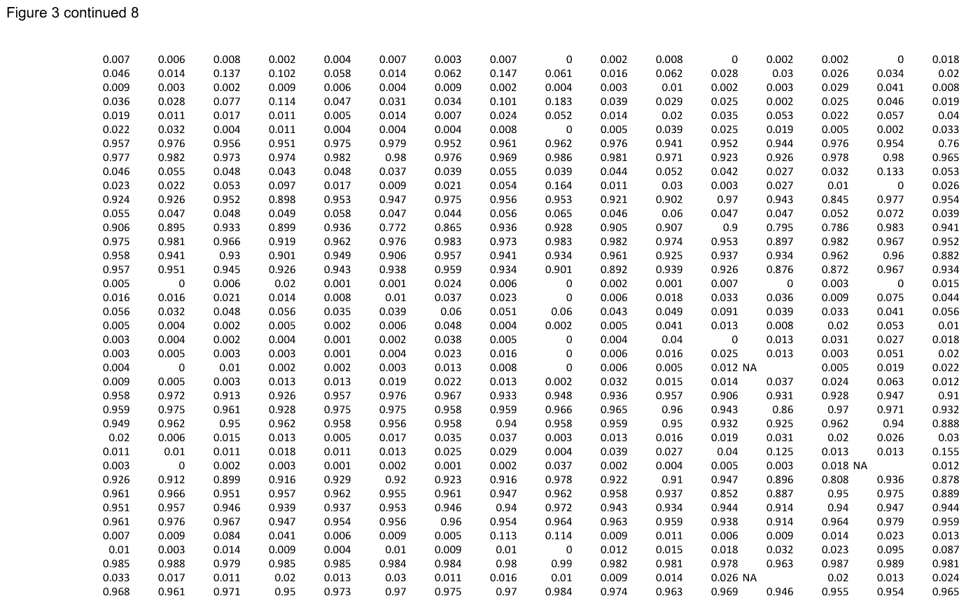

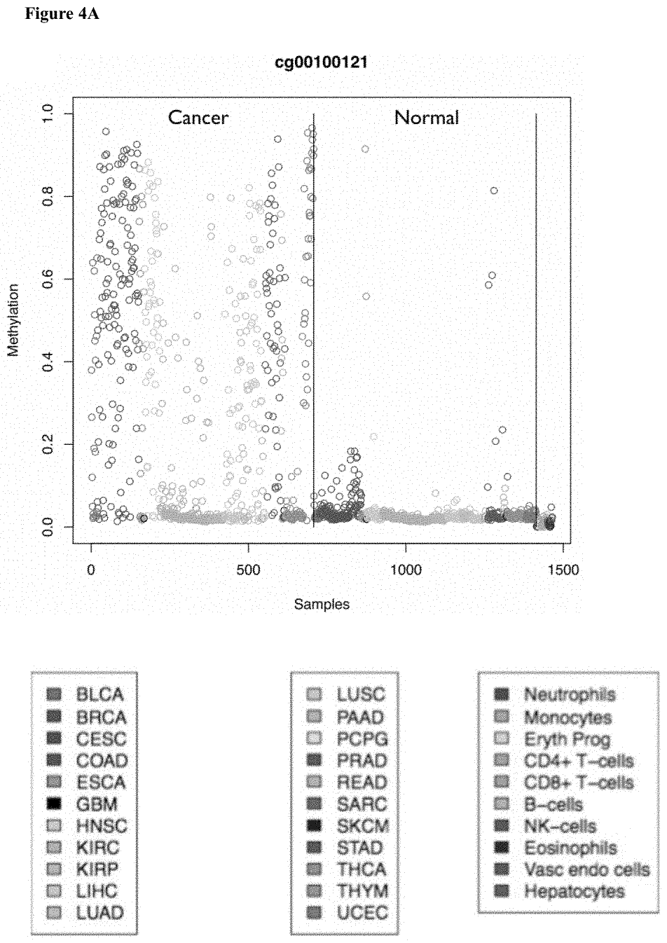

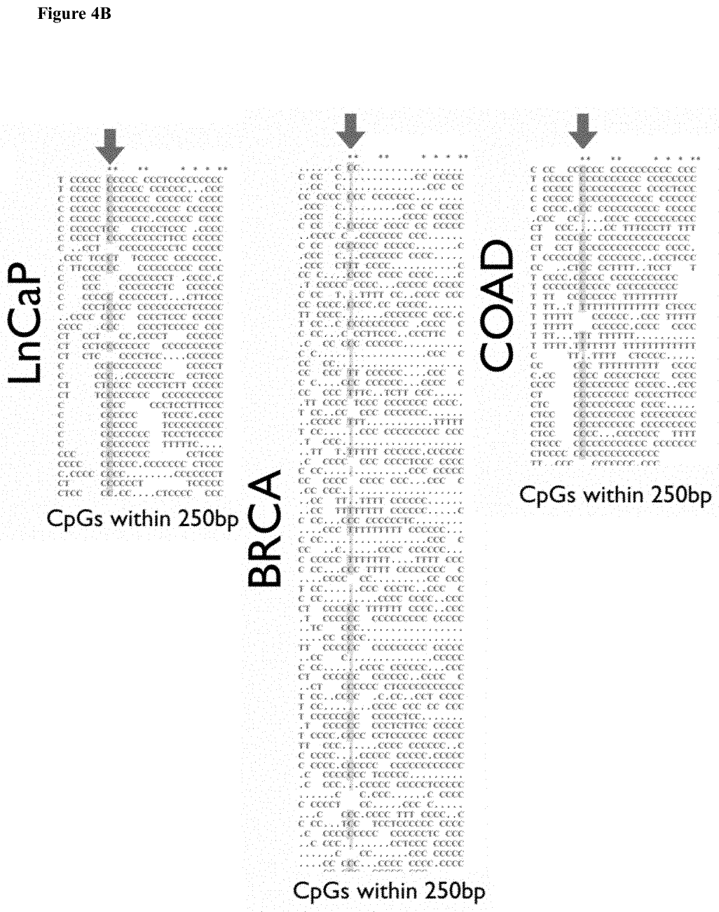

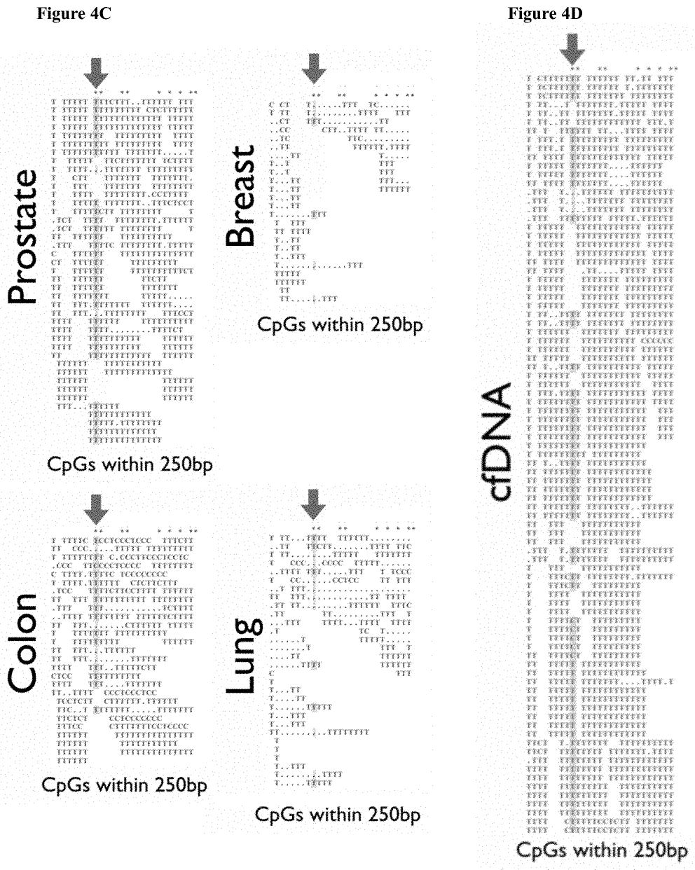

This analysis yielded 87 genomic regions that are differentially methylated in cancer. Each region comprised a central CpG whose methylation status was used to establish the region (250 nucleotides upstream and 250 downstream from the central CpG). Other CpGs within the region followed the same cancer specific methylation pattern as the central CpG (see FIGS. 4-6). Regions with cancer specific hypermethylation are provided in Table 1. Regions with cancer specific hypomethylation are provided in Table 2. FIG. 3 provides Table 3 which summarizes the methylation status of the 87 regions in 32 cancers, 23 matched healthy samples from the cancers and 34 healthy tissues/cell types. Table 3 also provides the average methylation value in cancer and in healthy tissue. Regions with a higher average in cancer are the hypermethylation regions and regions with lower average in cancer are the hypomethylated regions. FIG. 2 provides a visual representation of the methylation values for eight of the regions provided in Table 3; seven of the regions show cancer specific hypermethylation and the eighth region shows cancer specific hypomethylation. The healthy cells types shown are the those whose DNA is most prevalent in blood cfDNA. As can be seen, not every marker/region is alternatively methylated in every cancer, but when the cancer-specific signal does appear it strongly indicates the presence of a cancerous cell. The methylation readings in healthy tissues are very consistent across many tissues and cell types.

TABLE 1
Hypermethylated regions in cancer

| | | | | Central | | |
|---|---|---|---|---|---|---|
| | | Region | Region | CpG | | |
| Chr | Region | Start | End | position | CpG # | Gene |
| chr1 | 1 | 33358707 | 33359207 | 33358957 | cg05660436 | HPCA |
| chr1 | 2 | 39956557 | 39957057 | 39956807 | cg04923576 | BMP8A |
| chr1 | 3 | 46632446 | 46632946 | 46632696 | cg27636310 | TSPAN1 |
| chr1 | 4 | 110672832 | 110673332 | 110673082 | cg01016662 | UBL4B |
| chr1 | 5 | 169396385 | 169396885 | 169396635 | cg00100121 | C1orf114 |
| chr1 | 6 | 169396456 | 169396956 | 169396706 | cg00002719 | C1orf114 |
| chr1 | 7 | 205424735 | 205425235 | 205424985 | cg14203032 | MIR135B |
| chr1 | 8 | 205424755 | 205425255 | 205425005 | cg15651650 | MIR135B |
| chr1 | 9 | 206681128 | 206681628 | 206681378 | cg18328206 | RASSF5 |
| chr1 | 10 | 232941003 | 232941503 | 232941253 | cg15542798 | MAP10 |
| chr10 | 11 | 43892765 | 43893265 | 43893015 | cg05525499 | HNRNPF |
| chr11 | 17 | 19735451 | 19735951 | 19735701 | cg20686479 | NAV2 |
| chr11 | 19 | 63381797 | 63382297 | 63382047 | cg15219506 | PLA2G16 |
| chr11 | 20 | 72463174 | 72463674 | 72463424 | cg03713592 | ARAP1 |
| chr12 | 22 | 99139518 | 99140018 | 99139768 | cg12391352 | ANKS1B |
| chr12 | 23 | 107297301 | 107297801 | 107297551 | cg16848054 | C12orf23 |
| chr13 | 25 | 20531369 | 20531869 | 20531619 | cg20880234 | ZMYM2 |
| chr13 | 26 | 23734054 | 23734554 | 23734304 | cg19356117 | SGCG |
| chr13 | 27 | 96204623 | 96205123 | 96204873 | cg10305311 | CLDN10 |
| chr15 | 28 | 67143441 | 67143941 | 67143691 | cg12317470 | SMAD6 |
| chr16 | 29 | 10276549 | 10277049 | 10276799 | cg16368442 | GRIN2A |
| chr17 | 30 | 6347541 | 6348041 | 6347791 | cg11090139 | FAM64A |
| chr17 | 31 | 6616633 | 6617133 | 6616883 | cg12146546 | SLC13A5 |
| chr17 | 32 | 35014162 | 35014662 | 35014412 | cg08967106 | MRM1 |
| chr17 | 33 | 36609524 | 36610024 | 36609774 | cg00755470 | ARHGAP23 |
| chr17 | 34 | 42092181 | 42092681 | 42092431 | cg12259256 | TMEM101 |
| chr17 | 35 | 54911893 | 54912393 | 54912143 | cg01344452 | C17orf67 |
| chr17 | 36 | 58498727 | 58499227 | 58498977 | cg09695735 | C17orf64 |
| chr18 | 37 | 8367125 | 8367625 | 8367375 | cg02996413 | LOC100192426 |
| chr19 | 38 | 3275663 | 3276163 | 3275913 | cg26825934 | CELF5 |
| chr19 | 39 | 13983563 | 13984063 | 13983813 | cg16005540 | MIR181C |
| chr19 | 40 | 14583029 | 14583529 | 14583279 | cg02782369 | PTGER1/PKN1 |
| chr19 | 41 | 17717051 | 17717551 | 17717301 | cg19027852 | UNC13A |
| chr19 | 43 | 42828371 | 42828871 | 42828621 | cg08371772 | TMEM145 |
| chr19 | 44 | 58281200 | 58281700 | 58281450 | cg14038484 | ZNF586 |
| chr2 | 46 | 87034581 | 87035081 | 87034831 | cg00670742 | CD8A |
| chr2 | 48 | 100938549 | 100939049 | 100938799 | cg23977631 | LONRF2 |
| chr2 | 49 | 201983169 | 201983669 | 201983419 | cg12049462 | CFLAR |
| chr2 | 50 | 228736209 | 228736709 | 228736459 | cg01216370 | DAW1 |
| chr2 | 51 | 232545616 | 232546116 | 232545866 | cg26008007 | PTMA |
| chr20 | 52 | 9495076 | 9495576 | 9495326 | cg20191310 | LAMP5 |
| chr22 | 53 | 18923626 | 18924126 | 18923876 | cg18713809 | PRODH |
| chr22 | 54 | 24110555 | 24111055 | 24110805 | cg12256538 | CHCHD10 |
| chr3 | 57 | 97690536 | 97691036 | 97690786 | cg24960158 | MINA |
| chr3 | 58 | 129694237 | 129694737 | 129694487 | cg08195943 | TRH |
| chr5 | 60 | 1386492 | 1386992 | 1386742 | cg11942971 | CLPTM1L |
| chr5 | 65 | 10333618 | 10334118 | 10333868 | cg24740026 | MARCH6 |
| chr5 | 66 | 95297791 | 95298291 | 95298041 | cg11571761 | ELL2 |
| chr6 | 67 | 26043970 | 26044470 | 26044220 | cg07701237 | HIST1H2BB |
| chr6 | 68 | 30711777 | 30712277 | 30712027 | cg27449131 | FLOT1 |
| chr6 | 69 | 30711808 | 30712308 | 30712058 | cg10938374 | FLOT1 |
| chr6 | 70 | 30712057 | 30712557 | 30712307 | cg20650802 | FLOT1 |
| chr6 | 71 | 30712123 | 30712623 | 30712373 | cg01665212 | FLOT1 |
| chr6 | 72 | 33160012 | 33160512 | 33160262 | cg13586420 | COL11A2 |
| chr7 | 76 | 139930006 | 139930506 | 139930256 | cg08042316 | LOC100134229 |
| chr7 | 77 | 149119431 | 149119931 | 149119681 | cg26269703 | ZNF777 |
| chr7 | 78 | 149470570 | 149471070 | 149470820 | cg18989174 | ZNF467 |
| chr8 | 83 | 11204916 | 11205416 | 11205166 | cg05362548 | TDH |
| chr8 | 84 | 21906496 | 21906996 | 21906746 | cg23967540 | FGF17 |
| chr9 | 86 | 4741460 | 4741960 | 4741710 | cg00958854 | AK3 |

TABLE 2
Hypomethylated regions in cancer

| | | | | Central | | |
|---|---|---|---|---|---|---|
| | | Region | Region | CpG | | |
| Chr | Region | Start | End | position | CpG # | Gene |
| chr10 | 12 | 124668644 | 124669144 | 124668894 | cg14440102 | FAM24A |
| chr10 | 13 | 133058351 | 133058851 | 133058601 | cg07810282 | TCERG1L |
| chr10 | 14 | 134683461 | 134683961 | 134683711 | cg23123895 | TTC40 |
| chr10 | 15 | 135153687 | 135154187 | 135153937 | cg17247026 | CALY |
| chr10 | 16 | 135153711 | 135154211 | 135153961 | cg24748548 | CALY |
| chr11 | 18 | 50237857 | 50238357 | 50238107 | cg24205065 | LOC441601 |
| chr11 | 21 | 94300251 | 94300751 | 94300501 | cg05907238 | PIWIL4 |
| chr12 | 24 | 132896493 | 132896993 | 132896743 | cg21167716 | GALNT9 |
| chr19 | 42 | 33622616 | 33623116 | 33622866 | cg14093289 | WDR88 |
| chr2 | 45 | 1878740 | 1879240 | 1878990 | cg17187595 | MYT1L |
| chr2 | 47 | 89371753 | 89372253 | 89372003 | cg05289966 | MIR4436A |
| chr3 | 55 | 70048582 | 70049082 | 70048832 | cg26680097 | MITF |
| chr3 | 56 | 94656689 | 94657189 | 94656939 | cg01954930 | LOC255025 |
| chr5 | 59 | 1363649 | 1364149 | 1363899 | cg16035036 | CLPTM1L |
| chr5 | 61 | 1442673 | 1443173 | 1442923 | cg04073265 | SLC6A3 |
| chr5 | 62 | 1950532 | 1951032 | 1950782 | cg00327669 | IRX4 |
| chr5 | 63 | 2633363 | 2633863 | 2633613 | cg26718232 | IRX2 |
| chr5 | 64 | 5025553 | 5026053 | 5025803 | cg18746831 | LOC340094 |
| chr7 | 73 | 1783699 | 1784199 | 1783949 | cg19266396 | ELFN1 |
| chr7 | 74 | 63652533 | 63653033 | 63652783 | cg20458740 | ZNF735 |
| chr7 | 75 | 139255997 | 139256497 | 139256247 | cg11355603 | HIPK2 |
| chr7 | 79 | 157869576 | 157870076 | 157869826 | cg10731951 | PTPRN2 |
| chr7 | 80 | 158549991 | 158550491 | 158550241 | cg18651659 | ESYT2 |
| chr7 | 81 | 158550028 | 158550528 | 158550278 | cg01987065 | ESYT2 |
| chr8 | 82 | 2538055 | 2538555 | 2538305 | cg15239628 | MYOM2 |
| chr8 | 85 | 59058985 | 59059485 | 59059235 | cg08274876 | FAM110B |
| chr9 | 87 | 99259206 | 99259706 | 99259456 | cg14160020 | HABP4 |

The regions around the central CpG were also investigated. In the majority of cancers and healthy samples the CpGs in the same block as the central CpG shared the same methylation pattern. This was observed in regions 100 nucleotides upstream and downstream of the central CpG and even as far out as 250 nucleotides upstream and downstream. FIGS. 4-6 show three regions in detail, including the methylation status of all cytosines in CpG dinucleotides within the 501-nucleotide region surrounding the central cytosine. Not every nucleotide from the region was sequenced in every read, and often the sheared DNA only partially covered the region. Reads that include the central CpG were included in the analysis.

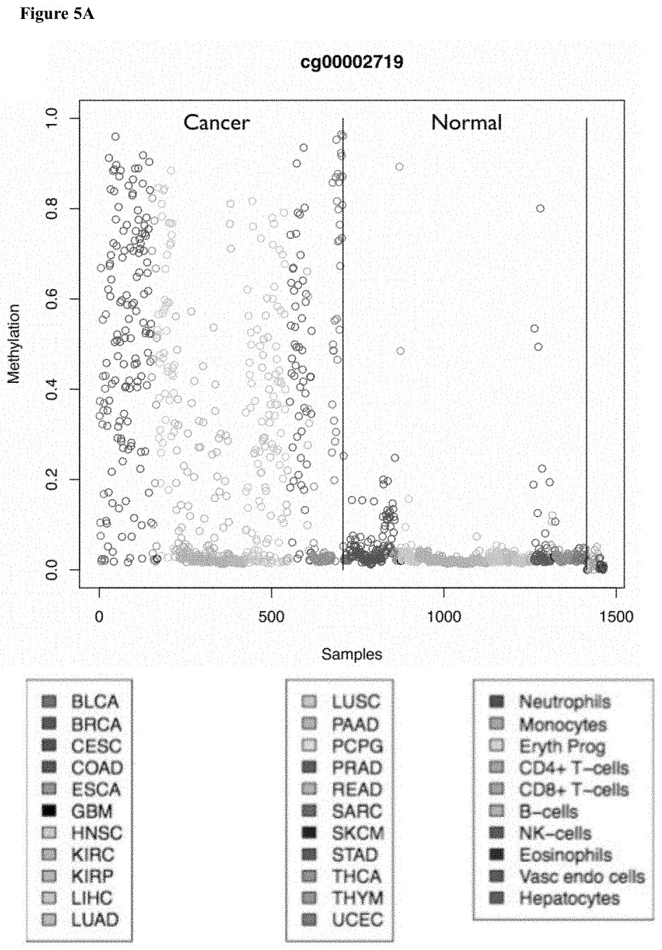

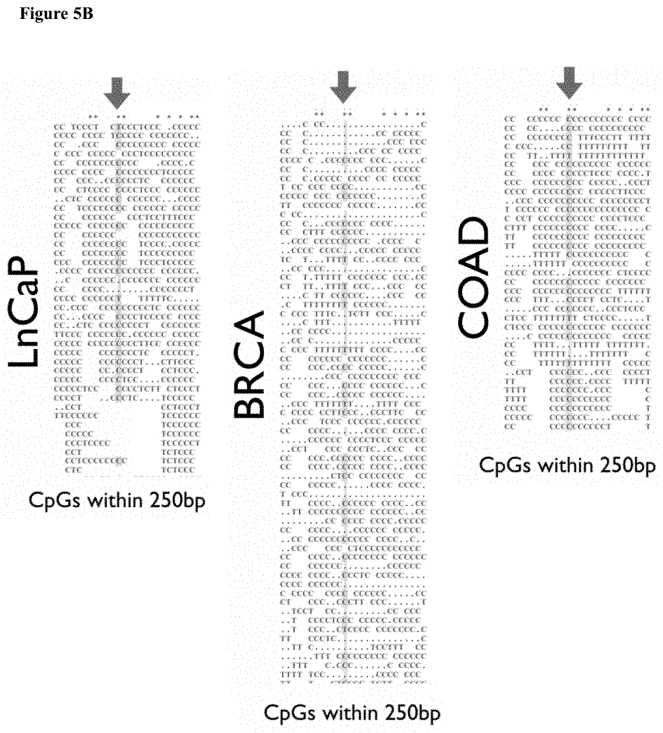

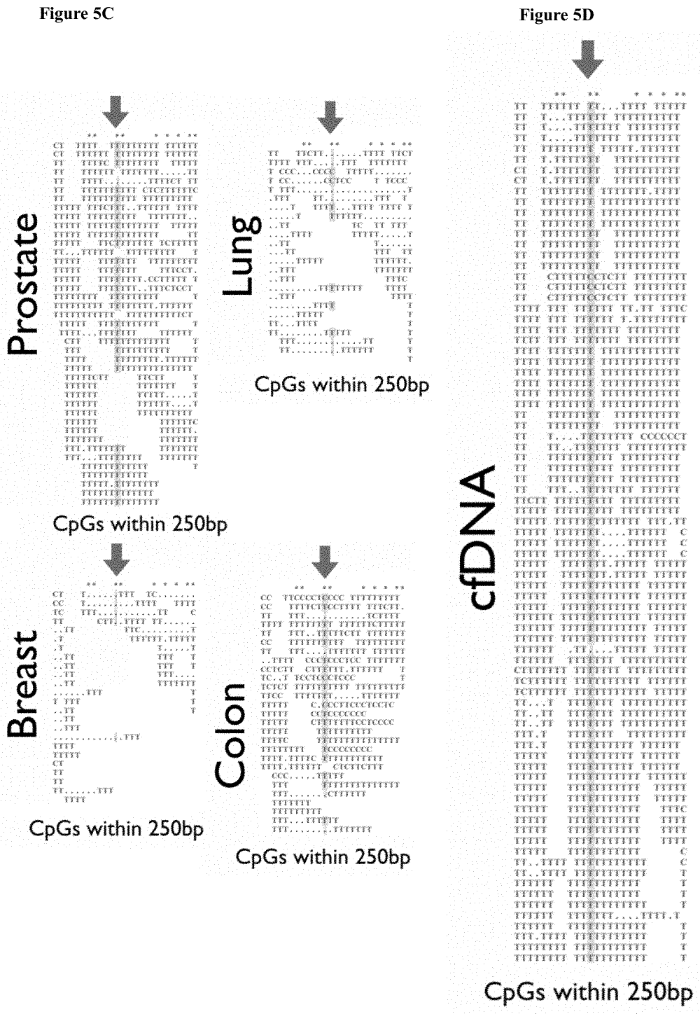

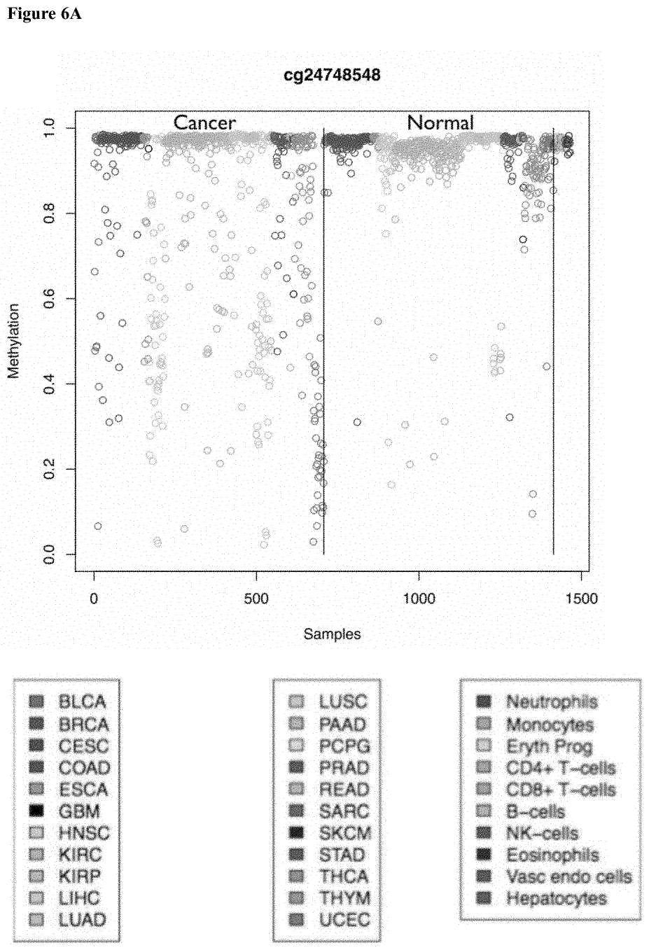

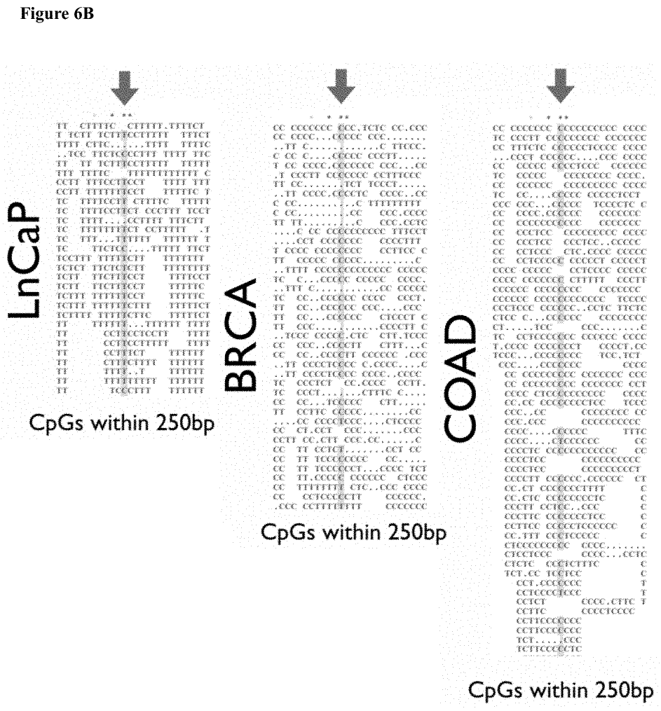

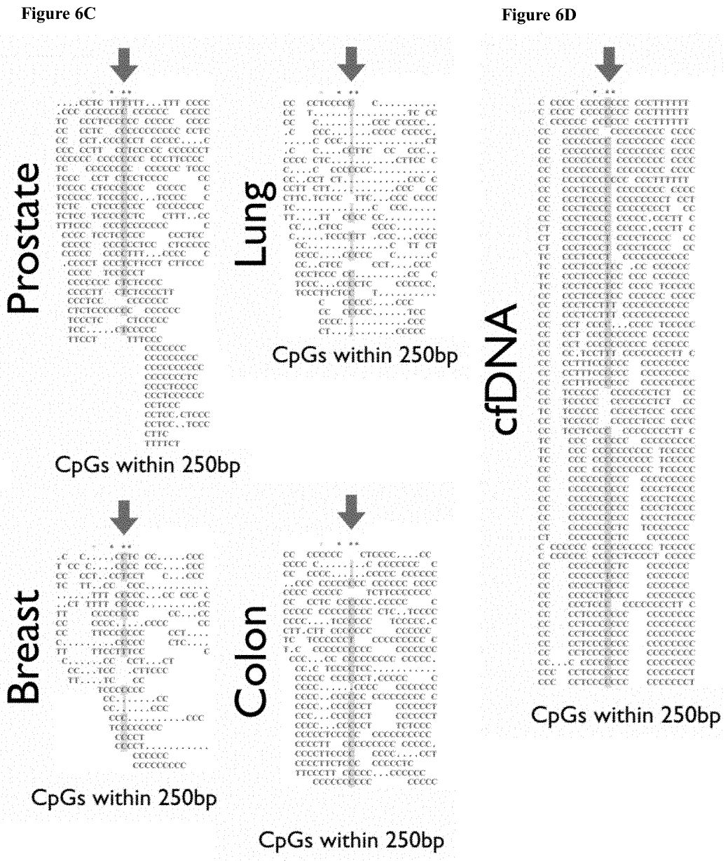

FIG. 4 shows a region hypermethylated in cancer, although there is heterogeneity between cancers (FIG. 4A). Even within an individual cancer type there is considerable heterogeneity, though the methylation pattern of the central CpG is highly conserved (FIG. 4B). Hypomethylation was observed in healthy tissues (FIG. 4C) and in cfDNA from healthy subjects (FIG. 4D) broadly throughout the region and most consistently at the central CpG. FIGS. 5A-D show another region that is differentially methylated (FIG. 5A), with hypermethylation in cancer (FIG. 5B), and hypomethylation in healthy tissue (FIG. 5C) and cfDNA from healthy subjects (FIG. 5D). FIG. 6 shows a region hypomethylated in cancer, which also shows heterogeneity between cancer types (FIG. 6A), and within cancer types (FIG. 6B). Although the hypomethylation signature was only observed in some cancers, the hypermethylation was very consistent across healthy tissues (FIG. 6C) and cfDNA samples (FIG. 6D).

### Example 3: Patient Sample Analysis

Next the predictive value of the marker regions was tested in patient samples. Tumor samples were surgically removed from cancer patients and stored as formalin-fixed and paraffin-embedded (FFPE) tissue blocks. Similarly, healthy tissue was also selected. DNA was extracted from the various samples using QIAamp DNA FFPE Tissue Kit and then treated with bisulfite. PCR was performed using primers specific to 13 markers (8 methylated in cancer: NAV2, TRH, HIST1H2BB, Cg10305311, Cg02996413, Cg01016662, Cg00755470, Cg00002719; and 5 unmethylated in cancer: MYT1L, Cg23123895, Cg24748548, Cg18746831, Cg17247026). The primers were specifically designed to bind regardless of potential methylation. PCR products were sequenced and the percentage of unmethylated and methylated molecules from all reads was calculated. Cancer specific methylation patterns were observed in all cancer samples at high levels. Individual markers showed a low number of reads in some healthy samples, and some markers were not present or lowly present in some cancers. However, when all the markers were combined every cancer sample had a higher number of cancer specific reads than every healthy sample (FIG. 7A).

Next, cfDNA samples were examined for cancer specific methylation patterns. CfDNA was extracted from plasma samples of patients with breast cancer and from plasma samples of healthy women. The cfDNA was treated with bisulfite and PCR using primers specific to 27 markers (15 methylated in cancer: Cg14203032, Cg02782369, Cg27636310, C17orf64, Cg16368442, Cg08042316, Cg08042316, HNRNPF, Cg01016662, NAV2, Cg19356117, Cg10305311, Cg00002719, Slc13a5, Cg14038484, ZMYM2, TRH; and 11 unmethylated in cancer: Cg26680097, ELFN, GALNT9, Cg05289966, Cg14160020, TCERG1, Cg17247026, Cg20458740, Cg00327669, Cg23123895, Cg26718232). PCR products were sequenced and the number of unmethylated molecules was calculated. The primers were specifically designed to bind regardless of potential methylation. PCR products were sequenced and the percentage of unmethylated and methylated molecules from all reads was calculated. Cancer specific methylation patterns were observed in all cancer samples accept two. Individual markers showed a low number of reads in some healthy samples, and some markers were not present or lowly present in some cancers. However, when all the markers were combined every cancer sample but one (PL3792) had a higher number of cancer specific reads than every healthy sample (FIG. 7B).

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

1. A method of detecting DNA from a cancerous cell in a sample, the method comprising:
a. receiving DNA methylation measurements of DNA from a sample in at least one genomic region comprising CpG dinucleotides, wherein said at least one genomic region is selected from a region provided in Table 1 and Table 2; and
b. assigning a sample as comprising DNA from a cancerous cell when said region comprises a cancer-specific methylation pattern;
thereby detecting DNA from a cancerous cell is a sample comprising DNA.

2. The method of claim 1, wherein
a. said receiving comprises providing a sample comprising DNA and measuring DNA methylation of the DNA in said at least one genomic region selected from a region provided in Table 1 and Table 2;
b. said sample is selected from a blood sample, a bodily fluid sample, a tissue sample and a tumor sample
c. said sample is a bodily fluid sample, and said DNA is cell-free DNA;
d. said sample is a bodily fluid sample and said biological fluid is selected from blood, plasma, serum, urine, feces, cerebral spinal fluid, lymph, tumor fluid and breast milk;
e. said sample is a bodily fluid sample and said providing comprises providing a bodily fluid and isolating said cfDNA from said bodily fluid; and
f. a combination thereof.

3. (canceled)

4. (canceled)

5. (canceled)

6. (canceled)

7. The method of claim 2, wherein said DNA is cfDNA and said cfDNA from a cancerous cell is less than 0.1% of said cfDNA.

8. The method of claim 1, wherein said sample is obtained from a subject and the method is for detecting cancer in said subject.

9. The method of claim 8, further comprising administering an anti-cancer therapy to a subject for whom cancer is detected.

10. The method of claim 1, wherein said measurements of DNA methylation comprises measurement of bisulfite converted DNA, performing a methylome array or chip on bisulfite converted DNA, sequencing bisulfite converted DNA, or are from performing methylation specific PCR.

11. (canceled)

12. (canceled)

13. The method of claim 1, wherein said cancer-specific methylation pattern is hypermethylation of at least one genomic region provided in Table 1 or hypomethylation of at least one region provided in Table 2.

14. The method of claim 1, wherein said cancer-specific methylation pattern is methylation of a central CpG of said at least one genomic region provided in Table 1 or unmethylation of a central CpG of said at least one genomic region provided in Table 2.

15. The method of claim 14, wherein said cancer-specific methylation pattern is methylation of a central CpG and further comprises methylation of at least one other CpG of said at least one genomic region.

16. The method of claim 13, wherein said hypermethylation comprises methylation of at least 5 CpGs within said region or said hypomethylation comprises unmethylation of at least 5 CpGs within said region.

17. (canceled)

18. (canceled)

19. The method of claim 14, wherein said cancer-specific methylation pattern is unmethylation of a central CpG and further comprises unmethylation of at least one other CpG of said at least one genomic region.

20. (canceled)

21. The method of claim 1, wherein said at least one region is a region from 100 nucleotides upstream of a central CpG provided in Table 1 and 2 to 100 nucleotides downstream of said central CpG.

22. The method of claim 1, wherein said cancer is selected from breast cancer, cervical cancer, endocervical cancer, colon cancer, lymphoma, esophageal cancer, brain cancer, head and neck cancer, renal cancer, meningeal cancer, glioma, glioblastoma, Langerhans cell cancer, lung cancer, mesothelioma, ovarian cancer, pancreatic cancer, neuroendocrine cancer, prostate cancer, skin cancer, stomach cancer, tenosynovial cancer, thyroid cancer, uterine cancer, and testicular cancer.

23. The method of claim 22, wherein said cancer-specific methylation pattern is a specific cancer-specific methylation pattern, and the cancer and region match based on the methylation levels provided in Table 3.

24. An array, consisting of methylation specific oligonucleotides reverse complementary to a sequence of a genomic region provided in Table 1 and Table 2 and comprising at least 10 methylation specific oligonucleotides; and optionally a solid support, wherein said at least 10 methylation specific oligonucleotides are immobilized to said solid support.

25. (canceled)

26. The array of claim 24, wherein a methylation specific oligonucleotide
a. only hybridizes in the presence of methylation or only binds in the absence of methylation;
b. is reverse complementary to a sequence of a region from Table 1 and is not complementary to sequence of a region from Table 1 wherein a cytosine of a CpG residue is converted to a thymine;
c. is reverse complementary to a sequence of a region from Table 2 wherein a cytosine of a CpG residue is converted to a thymine and is not complementary to a sequence of a region from Table 2; or
d. a combination thereof.

27. (canceled)

28. The array of claim 24, comprising
a. at least 100 oligonucleotides;
b. a plurality of oligonucleotide that are reverse complementary to a region;
c. at least one methylation specific oligonucleotide reverse complementary to each region in Table 1 and Table 2.

29. (canceled)

30. (canceled)

31. The array claim 24, wherein a methylation specific oligonucleotide reverse complementary to a region is reverse complementary to a central CpG of said region.

32. The array of claim 24, wherein said methylation specific oligonucleotide is reverse complementary to a region from 100 nucleotides upstream of a central CpG provided in Table 1 and Table 2 to 100 nucleotides downstream of said central CpG.

33. A kit comprising an array of claim 24 and at least one reagent for amplification of a target DNA molecule hybridized to an oligonucleotide of said array, optionally wherein said reagent is selected from a polymerase, a forward primer, a reverse primer, an adapter, and a pool of free nucleotides.

34. (canceled)


SEQUENCE LISTING (118 sequences)

SEQ ID NO: 1 - DNA, 20 bp, Artificial
  Synthetic
gttgatgtttgttatagggt

SEQ ID NO: 2 - DNA, 22 bp, Artificial
  Synthetic
tatatatccaaaaaaccaaccc

SEQ ID NO: 3 - DNA, 20 bp, Artificial
  Synthetic
ttagggaagaaaaggtggtt

SEQ ID NO: 4 - DNA, 20 bp, Artificial
  Synthetic
aaaaatactcaaaaaacccc

SEQ ID NO: 5 - DNA, 24 bp, Artificial
  Synthetic
tattttttttgtttgtgtaaaatg

SEQ ID NO: 6 - DNA, 25 bp, Artificial
  Synthetic
ccataacaatataatcctaactacc

SEQ ID NO: 7 - DNA, 20 bp, Artificial
  Synthetic
ggttttttttttggtagtga

SEQ ID NO: 8 - DNA, 23 bp, Artificial
  Synthetic
attctataaacccctaactaaaa

SEQ ID NO: 9 - DNA, 22 bp, Artificial
  Synthetic
agtgaagttgaggtttttaagg

SEQ ID NO: 10 - DNA, 22 bp, Artificial
  Synthetic
aaaatttcacaaccaacacaac

SEQ ID NO: 11 - DNA, 20 bp, Artificial
  Synthetic
gagagaggtggttatggttg

SEQ ID NO: 12 - DNA, 24 bp, Artificial
  Synthetic
aaacatacacaacaaataacacac

SEQ ID NO: 13 - DNA, 20 bp, Artificial
  Synthetic
gttggaagggtgtaaggtgt

SEQ ID NO: 14 - DNA, 20 bp, Artificial
  Synthetic
aaaacactacacaatccccc

SEQ ID NO: 15 - DNA, 24 bp, Artificial
  Synthetic
aaggaagtttaggtgagataggtt

SEQ ID NO: 16 - DNA, 24 bp, Artificial
  Synthetic
ctccccctactactcctactctac

SEQ ID NO: 17 - DNA, 22 bp, Artificial
  Synthetic
ggaattgtatttattttggagg

SEQ ID NO: 18 - DNA, 25 bp, Artificial
  Synthetic
ctttaaaaataaaaaaccattctac

SEQ ID NO: 19 - DNA, 21 bp, Artificial
  Synthetic
atattttgggagatgagatgg

SEQ ID NO: 20 - DNA, 20 bp, Artificial
  Synthetic
tactaaacaaaacccctccc

SEQ ID NO: 21 - DNA, 25 bp, Artificial
  Synthetic
ggagaggatgatattattggtaata

SEQ ID NO: 22 - DNA, 25 bp, Artificial
  Synthetic
ctctcccaaaatattataaacaata

SEQ ID NO: 23 - DNA, 24 bp, Artificial
  Synthetic
gtgttaggagattaagttttgatt

SEQ ID NO: 24 - DNA, 26 bp, Artificial
  Synthetic
ctaaaaacttaccacaactaataaac

SEQ ID NO: 25 - DNA, 23 bp, Artificial
  Synthetic
agtaagagagggatagagatagg

SEQ ID NO: 26 - DNA, 24 bp, Artificial
  Synthetic
caaaaatctaaaaataacaaaaaa

SEQ ID NO: 27 - DNA, 20 bp, Artificial
  Synthetic
ggggaggtagtgatttaggt

SEQ ID NO: 28 - DNA, 21 bp, Artificial
  Synthetic
ccttaaaaaaaaaaccaaaac

SEQ ID NO: 29 - DNA, 22 bp, Artificial
  Synthetic
ggttgttagtttgaatttgagt

SEQ ID NO: 30 - DNA, 21 bp, Artificial
  Synthetic
ttctccatctacaactaaccc

SEQ ID NO: 31 - DNA, 24 bp, Artificial
  Synthetic
atagaaaggttgatgtttgttata

SEQ ID NO: 32 - DNA, 24 bp, Artificial
  Synthetic
accataaatatatatccaaaaaac

SEQ ID NO: 33 - DNA, 22 bp, Artificial
  Synthetic
gaggttgatagaagatagggag

SEQ ID NO: 34 - DNA, 23 bp, Artificial
  Synthetic
cccttactacataaaactaaacc

SEQ ID NO: 35 - DNA, 20 bp, Artificial
  Synthetic
ggagggtaaaggtttgtagg

SEQ ID NO: 36 - DNA, 22 bp, Artificial
  Synthetic
tcacacttctttcccaataaac

SEQ ID NO: 37 - DNA, 23 bp, Artificial
  Synthetic
tagggttaggagaaattattgtt

SEQ ID NO: 38 - DNA, 25 bp, Artificial
  Synthetic
aaaactctaataaaccaaatctatt

SEQ ID NO: 39 - DNA, 23 bp, Artificial
  Synthetic
ggtaaaattttttaaaaggaata

SEQ ID NO: 40 - DNA, 23 bp, Artificial
  Synthetic
aaacactcacctaaaaactaacc

SEQ ID NO: 41 - DNA, 26 bp, Artificial
  Synthetic
tttatgtttaggatattaatttattg

SEQ ID NO: 42 - DNA, 26 bp, Artificial
  Synthetic
ccataattcaataaaaataatattac

SEQ ID NO: 43 - DNA, 22 bp, Artificial
  Synthetic
gagtgggttattagggtttttt

SEQ ID NO: 44 - DNA, 24 bp, Artificial
  Synthetic
aaaaacaaaaactccaataatctt

SEQ ID NO: 45 - DNA, 21 bp, Artificial
  Synthetic
gggttgattttattttttgga

SEQ ID NO: 46 - DNA, 21 bp, Artificial
  Synthetic
cacacaaccattcaaaatcaa

SEQ ID NO: 47 - DNA, 18 bp, Artificial
  Synthetic
ggttggtgtgtttgaggg

SEQ ID NO: 48 - DNA, 19 bp, Artificial
  Synthetic
aaaaaaactacctttcccc

SEQ ID NO: 49 - DNA, 22 bp, Artificial
  Synthetic
ttatttattttgaggatggttt

SEQ ID NO: 50 - DNA, 21 bp, Artificial
  Synthetic
taaccacccacaactaaaaac

SEQ ID NO: 51 - DNA, 23 bp, Artificial
  Synthetic
attagtaagtgtgaaggtagggg

SEQ ID NO: 52 - DNA, 26 bp, Artificial
  Synthetic
ccaaaaattattatctccttatattc

SEQ ID NO: 53 - DNA, 23 bp, Artificial
  Synthetic
gaggtggtgagtgaatgtgttat

SEQ ID NO: 54 - DNA, 22 bp, Artificial
  Synthetic
aaaacttcattcctaaaaaccc

SEQ ID NO: 55 - DNA, 22 bp, Artificial
  Synthetic
aggagtgttatgttggaatttg

SEQ ID NO: 56 - DNA, 22 bp, Artificial
  Synthetic
cctctccaaaacaacctatatc

SEQ ID NO: 57 - DNA, 21 bp, Artificial
  Synthetic
ggtgatggatatggaaggatt

SEQ ID NO: 58 - DNA, 24 bp, Artificial
  Synthetic
acctatatccctctatatccttcc

SEQ ID NO: 59 - DNA, 29 bp, Artificial
  Synthetic
aagttaagttatagttatttttgttatat

SEQ ID NO: 60 - DNA, 24 bp, Artificial
  Synthetic
ccacaactactaacaaaacaaatc

SEQ ID NO: 61 - DNA, 18 bp, Artificial
  Synthetic
gggtgtttgggtggaaag

SEQ ID NO: 62 - DNA, 22 bp, Artificial
  Synthetic
ccactacaaataccacatcaaa

SEQ ID NO: 63 - DNA, 24 bp, Artificial
  Synthetic
aagaaagatttagtgggtataagg

SEQ ID NO: 64 - DNA, 25 bp, Artificial
  Synthetic
accataacactcacacctaataacc

SEQ ID NO: 65 - DNA, 23 bp, Artificial
  Synthetic
tattgtaattgttttggggtatt

SEQ ID NO: 66 - DNA, 22 bp, Artificial
  Synthetic
ctacaaaacaatcaaaacccac

SEQ ID NO: 67 - DNA, 25 bp, Artificial
  Synthetic
ggttttagttttgatatttaagaaa

SEQ ID NO: 68 - DNA, 21 bp, Artificial
  Synthetic
tacaacaaatacacaccccac

SEQ ID NO: 69 - DNA, 23 bp, Artificial
  Synthetic
agaaggaaataggagtgggagtt

SEQ ID NO: 70 - DNA, 20 bp, Artificial
  Synthetic
tcccaacaacccccaacaac

SEQ ID NO: 71 - DNA, 23 bp, Artificial
  Synthetic
tgttttgttttgttttgtttttt

SEQ ID NO: 72 - DNA, 25 bp, Artificial
  Synthetic
aacaaaacttacaataaaccaaaat

SEQ ID NO: 73 - DNA, 22 bp, Artificial
  Synthetic
ttatggatttaggtgaggatag

SEQ ID NO: 74 - DNA, 22 bp, Artificial
  Synthetic
tttataaacccaaattaaaaac

SEQ ID NO: 75 - DNA, 20 bp, Artificial
  Synthetic
tattttgagggggtggagtt

SEQ ID NO: 76 - DNA, 23 bp, Artificial
  Synthetic
taataactctacccccaaaacac

SEQ ID NO: 77 - DNA, 22 bp, Artificial
  Synthetic
aagattttggtttttttttttt

SEQ ID NO: 78 - DNA, 21 bp, Artificial
  Synthetic
aaaattaaaaataccttcccc

SEQ ID NO: 79 - DNA, 22 bp, Artificial
  Synthetic
ttaagggatagggtatgggtgt

SEQ ID NO: 80 - DNA, 20 bp, Artificial
  Synthetic
cactcccaacccccaaactc

SEQ ID NO: 81 - DNA, 21 bp, Artificial
  Synthetic
gtttttgtgtgttttgggtta

SEQ ID NO: 82 - DNA, 23 bp, Artificial
  Synthetic
aactaaaaataaaatttcccttc

SEQ ID NO: 83 - DNA, 23 bp, Artificial
  Synthetic
ggatttaggttatattgggatgt

SEQ ID NO: 84 - DNA, 25 bp, Artificial
  Synthetic
ataactccactaactcctcctactc

SEQ ID NO: 85 - DNA, 27 bp, Artificial
  Synthetic
ttatattaaatttattttatgtttagg

SEQ ID NO: 86 - DNA, 27 bp, Artificial
  Synthetic
atatccataattcaataaaaataatat

SEQ ID NO: 87 - DNA, 26 bp, Artificial
  Synthetic
gaaaattaaagattttagttgttaat

SEQ ID NO: 88 - DNA, 26 bp, Artificial
  Synthetic
actataaaaaaactcctaaacttaac

SEQ ID NO: 89 - DNA, 21 bp, Artificial
  Synthetic
gtgtgtgtgtgagtgtgggag

SEQ ID NO: 90 - DNA, 22 bp, Artificial
  Synthetic
aaactaacccaacaaccaaaaa

SEQ ID NO: 91 - DNA, 26 bp, Artificial
  Synthetic
gttattttagtttgtttgttttttat

SEQ ID NO: 92 - DNA, 23 bp, Artificial
  Synthetic
tttactttaactccattttccac

SEQ ID NO: 93 - DNA, 24 bp, Artificial
  Synthetic
ggggaaagtttagagtgttagtta

SEQ ID NO: 94 - DNA, 26 bp, Artificial
  Synthetic
cataatcaaatatacaaaccaaaata

SEQ ID NO: 95 - DNA, 25 bp, Artificial
  Synthetic
gtttagtaggtattttagaaggaag

SEQ ID NO: 96 - DNA, 24 bp, Artificial
  Synthetic
aacaccctctactctcaactactc

SEQ ID NO: 97 - DNA, 22 bp, Artificial
  Synthetic
ggggtagttttttttttatttt

SEQ ID NO: 98 - DNA, 28 bp, Artificial
  Synthetic
tatacatactaaaatattccataaaacc

SEQ ID NO: 99 - DNA, 25 bp, Artificial
  Synthetic
ggaagatattgattgagtatagagt

SEQ ID NO: 100 - DNA, 23 bp, Artificial
  Synthetic
aaaatatcactataacctttccc

SEQ ID NO: 101 - DNA, 20 bp, Artificial
  Synthetic
ggttagggaagggaattatt

SEQ ID NO: 102 - DNA, 22 bp, Artificial
  Synthetic
aaaactcttaaaacaaacctcc

SEQ ID NO: 103 - DNA, 21 bp, Artificial
  Synthetic
atatagggttttgtttgggtt

SEQ ID NO: 104 - DNA, 22 bp, Artificial
  Synthetic
accctaaactaaacccaaatac

SEQ ID NO: 105 - DNA, 24 bp, Artificial
  Synthetic
gtttgtttgttttatgagaggtta

SEQ ID NO: 106 - DNA, 24 bp, Artificial
  Synthetic
tcttataaacctctcttaaatccc

SEQ ID NO: 107 - DNA, 20 bp, Artificial
  Synthetic
tttgggttggtgtgatttgt

SEQ ID NO: 108 - DNA, 26 bp, Artificial
  Synthetic
cctaccttcacacttactaatacaac

SEQ ID NO: 109 - DNA, 22 bp, Artificial
  Synthetic
gtttgtttttaattttgttatt

SEQ ID NO: 110 - DNA, 22 bp, Artificial
  Synthetic
caaactacccctaaaaaactaa

SEQ ID NO: 111 - DNA, 22 bp, Artificial
  Synthetic
atgggtgttaaggttaggaagt

SEQ ID NO: 112 - DNA, 21 bp, Artificial
  Synthetic
cttaaaataaccaacaacccc

SEQ ID NO: 113 - DNA, 21 bp, Artificial
  Synthetic
tttagaggtgataggtgtgga

SEQ ID NO: 114 - DNA, 20 bp, Artificial
  Synthetic
caaaaacaaaaccactaccc

SEQ ID NO: 115 - DNA, 27 bp, Artificial
  Synthetic
tatttttagttgtaattttattaagaa

SEQ ID NO: 116 - DNA, 22 bp, Artificial
  Synthetic
aaaatcttctcttttattcctc

SEQ ID NO: 117 - DNA, 23 bp, Artificial
  Synthetic
ttgttttggatatttagttgatg

SEQ ID NO: 118 - DNA, 23 bp, Artificial
  Synthetic
atatcacacttctttcccaataa